SEQUENCE LISTING (6 sequences)

SEQ ID NO: 1 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - DNA, 1296 bp, Homo sapiens
atgcacgtgcgctcactgcgagctgcggcgccgcacagcttcgtggcgctctgggcacccctgttcctgctgcgctccgccctggccgacttcagcctggacaacgaggtgcactcgagcttcatccaccggcgcctccgcagccaggagcggcgggagatgcagcgcgagatcctctccattttgggcttgccccaccgcccgcgcccgcacctccagggcaagcacaactcggcacccatgttcatgctggacctgtacaacgccatggcggtggaggagggcggcgggcccggcggccagggcttctcctacccctacaaggccgtcttcagtacccagggcccccctctggccagcctgcaagatagccatttcctcaccgacgccgacatggtcatgagcttcgtcaacctcgtggaacatgacaaggaattcttccacccacgctaccaccatcgagagttccggtttgatctttccaagatcccagaaggggaagctgtcacggcagccgaattccggatctacaaggactacatccgggaacgcttcgacaatgagacgttccggatcagcgtttatcaggtgctccaggagcacttgggcagggaatcggatctcttcctgctcgacagccgtaccctctgggcctcggaggagggctggctggtgtttgacatcacagccaccagcaaccactgggtggtcaatccgcggcacaacctgggcctgcagctctcggtggagacgctggatgggcagagcatcaaccccaagttggcgggcctgattgggcggcacgggccccagaacaagcagcccttcatggtggctttcttcaaggccacggaggtccacttccgcagcatccggtccacggggagcaaacagcgcagccagaaccgctccaagacgcccaagaaccaggaagccctgcggatggccaacgtggcagagaacagcagcagcgaccagaggcaggcctgtaagaagcacgagctgtatgtcagcttccgagacctgggctggcaggactggatcatcgcgcctgaaggctacgccgcctactactgtgagggggagtgtgccttccctctgaactcctacatgaacgccaccaaccacgccatcgtgcagacgctggtccacttcatcaacccggaaacggtgcccaagccctgctgtgcgcccacgcagctcaatgccatctccgtcctctacttcgatgacagctccaacgtcatcctgaagaaatacagaaacatggtggtccgggcctgtggctgccactag

SEQ ID NO: 3 - protein, 1503 aa, Homo sapiens
MAGVGAAALSLLLHLGALALAAGAEGGAVPREPPGQQTTAHSSVLAGNSQEQWHPLREWLGRLEAAVMELREQNKDLQTRVRQLESCECHPASPQCWGLGRAWPEGARWEPDACTACVCQDGAAHCGPQAHLPHCRGCSQNGQTYGNGETFSPDACTTCRCLTGAVQCQGPSCSELNCLESCTPPGECCPICCTEGGSHWEHGQEWTTPGDPCRICRCLEGHIQCRQRECASLCPYPARPLPGTCCPVCDGCFLNGREHRSGEPVGSGDPCSHCRCANGSVQCEPLPCPPVPCRHPGKIPGQCCPVCDGCEYQGHQYQSQETFRLQERGLCVRCSCQAGEVSCEEQECPVTPCALPASGRQLCPACELDGEEFAEGVQWEPDGRPCTACVCQDGVPKCGAVLCPPAPCQHPTQPPGACCPSCDSCTYHSQVYANGQNFTDADSPCHACHCQDGTVTCSLVDCPPTTCARPQSGPGQCCPRCPDCILEEEVFVDGESFSHPRDPCQECRCQEGHAHCQPRPCPRAPCAHPLPGTCCPNDCSGCAFGGKEYPSGADFPHPSDPCRLCRCLSGNVQCLARRCVPLPCPEPVLLPGECCPQCPAPAGCPRPGAAHARHQEYFSPPGDPCRRCLCLDGSVSCQRLPCPPAPCAHPRQGPCCPSCDGCLYQGKEFASGERFPSPTAACHLCLCWEGSVSCEPKACAPALCPFPARGDCCPDCDGCEYLGESYLSNQEFPDPREPCNLCTCLGGFVTCGRRPCEPPGCSHPLIPSGHCCPTCQGCRYHGVTTASGETLPDPLDPTCSLCTCQEGSMRCQKKPCPPALCPHPSPGPCFCPVCHSCLSQGREHQDGEEFEGPAGSCEWCRCQAGQVSCVRLQCPPLPCKLQVTERGSCCPRCRGCLAHGEEHPEGSRWVPPDSACSSCVCHEGVVTCARIQCISSCAQPRQGPHDCCPQCSDCEHEGRKYEPGESFQPGADPCEVCICEPQPEGPPSLRCHRRQCPSLVGCPPSQLLPPGPQHCCPTCAEALSNCSEGLLGSELAPPDPCYTCQCQDLTWLCIHQACPELSCPLSERHTPPGSCCPVCRAPTQSCVHQGREVASGERWTVDTCTSCSCMAGTVRCQSQRCSPLSCGPDKAPALSPGSCCPRCLPRPASCMAFGDPHYRTFDGRLLHFQGSCSYVLAKDCHSGDFSVHVTNDDRGRSGVAWTQEVAVLLGDMAVRLLQDGAVTVDGHPVALPFLQEPLLYVELRGHTVILHAQPGLQVLWDGQSQVEVSVPGSYQGRTCGLCGNFNGFAQDDLQGPEGLLLPSEAAFGNSWQVSEGLWPGRPCSAGREVDPCRAAGYRARREANARCGVLKSSPFSRCHAVVPPEPFFAACVYDLCACGPGSSADACLCDALEAYASHCRQAGVTPTWRGPTLCVVGCPLERGFVFDECGPPCPRTCFNQHIPLGELAAHCVRPCVPGCQCPAGLVEHEAHCIPPEACPQVLLTGDQPLGARPSPSREPQETP

SEQ ID NO: 4 - DNA, 4733 bp, Homo sapiens
gagccgcgacgacagacggcgagccgagcgaggcggagctagcatggccggggtcggggccgctgcgctgtcccttctcctgcacctcggggccctggcgctggccgcgggcgcggaaggtggggctgtccccagggagccccctgggcagcagacaactgcccattcctcagtccttgctgggaactcccaggagcagtggcaccccctgcgagagtggctggggcgactggaggctgcagtgatggagctcagagaacagaataaggacctgcagacgagggtgaggcagctggagtcctgtgagtgccaccctgcatctccccagtgctgggggctggggcgtgcctggcccgagggggcacgctgggagcctgacgcctgcacagcctgcgtctgccaggatggggccgctcactgtggcccccaagcacacctgccccattgcaggggctgcagccaaaatggccagacctacggcaacggggagaccttctccccagatgcctgcaccacctgccgctgtctgacaggagccgtgcagtgccaggggccctcgtgttcagagctcaactgcttggagagctgcaccccacctggggagtgctgccccatctgctgcacagaaggtggctctcactgggaacatggccaagagtggacaacacctggggacccctgccgaatctgccggtgcctggagggtcacatccagtgccgccagcgagaatgtgccagcctgtgtccatacccagcccggcccctcccaggcacctgctgccctgtgtgtgatggctgtttcctaaacgggcgggagcaccgcagcggggagcctgtgggctcaggggacccctgctcgcactgccgctgtgctaatgggagtgtccagtgtgagcctctgccctgcccgccagtgccctgcagacacccaggcaagatccctgggcagtgctgccctgtctgcgatggctgtgagtaccagggacaccagtatcagagccaggagaccttcagactccaagagcggggcctctgtgtccgctgctcctgccaggctggcgaggtctcctgtgaggagcaggagtgcccagtcaccccctgtgccctgcctgcctctggccgccagctctgcccagcctgtgagctggatggagaggagtttgctgagggagtccagtgggagcctgatggtcggccctgcaccgcctgcgtctgtcaagatggggtacccaagtgcggggctgtgctctgccccccagccccctgccagcaccccacccagccccctggtgcctgctgccccagctgtgacagctgcacctaccacagccaagtgtatgccaatgggcagaacttcacggatgcagacagcccttgccatgcctgccactgtcaggatggaactgtgacatgctccttggttgactgccctcccacgacctgtgccaggccccagagtggaccaggccagtgttgccccaggtgcccagactgcatcctggaggaagaggtgtttgtggacggcgagagcttctcccacccccgagacccctgccaggagtgccgatgccaggaaggccatgcccactgccagcctcgcccctgccccagggccccctgtgcccacccgctgcctgggacctgctgcccgaacgactgcagcggctgtgcctttggcgggaaagagtaccccagcggagcggacttcccccacccctctgacccctgccgtctgtgtcgctgtctgagcggcaacgtgcagtgcctggcccgccgctgcgtgccgctgccctgtccagagcctgtcctgctgccgggagagtgctgcccgcagtgcccagcccccgccggctgcccacggcccggcgcggcccacgcccgccaccaggagtacttctccccgcccggcgatccctgccgccgctgcctctgcctcgacggctccgtgtcctgccagcggctgccctgcccgcccgcgccctgcgcgcacccgcgccaggggccttgctgcccctcctgcgacggctgcctgtaccaggggaaggagtttgccagcggggagcgcttcccatcgcccactgctgcctgccacctctgcctttgctgggagggcagcgtgagctgcgagcccaaggcatgtgcccctgcactgtgccccttccctgccaggggcgactgctgccctgactgtgatggctgtgagtacctgggggagtcctacctgagtaaccaggagttcccagacccccgagaaccctgcaacctgtgtacctgtcttggaggcttcgtgacctgcggccgccggccctgtgagcctccgggctgcagccacccactcatcccctctgggcactgctgcccgacctgccagggatgccgctaccatggcgtcactactgcctccggagagacccttcctgacccacttgaccctacctgctccctctgcacctgccaggaaggttccatgcgctgccagaagaagccatgtcccccagctctctgcccccacccctctccaggcccctgcttctgccctgtttgccacagctgtctctctcagggccgggagcaccaggatggggaggagtttgagggaccagcaggcagctgtgagtggtgtcgctgtcaggctggccaggtcagctgtgtgcggctgcagtgcccaccccttccctgcaagctccaggtcaccgagcgggggagctgctgccctcgctgcagaggctgcctggctcatggggaagagcaccccgaaggcagtagatgggtgccccccgacagtgcctgctcctcctgtgtgtgtcacgagggcgtcgtcacctgtgcacgcatccagtgcatcagctcttgcgcccagccccgccaagggccccatgactgctgtcctcaatgctctgactgtgagcatgagggccggaagtacgagcctggggagagcttccagcctggggcagacccctgtgaagtgtgcatctgcgagccacagcctgaggggcctcccagccttcgctgtcaccggcggcagtgtcccagcctggtgggctgcccccccagccagctcctgccccctgggccccagcactgctgtcccacctgtgccgaggccttgagtaactgttcagagggcctgctgggatctgagctagccccaccagacccctgctacacgtgccagtgccaggacctgacatggctctgcatccaccaggcttgtcctgagctcagctgtcccctctcagagcgccacactccccctgggagctgctgccccgtatgccgggctcccacccagtcctgcgtgcaccagggccgtgaggtggcctctggagagcgctggactgtggacacctgcaccagctgctcctgcatggcgggcaccgtgcgttgccagagccagcgctgctcaccgctctcgtgtggccccgacaaggcccctgccctgagtcctggcagctgctgcccccgctgcctgcctcggcccgcttcctgcatggccttcggagacccccattaccgcaccttcgacggccgcctgctgcacttccagggcagttgcagctatgtgctggccaaggactgccacagcggggacttcagtgtgcacgtgaccaatgatgaccggggccggagcggtgtggcctggacccaggaggtggcggtgctgctgggagacatggccgtgcggctgctgcaggacggggcagtcacggtggatgggcacccggtggccttgcccttcctgcaggagccgctgctgtatgtggagctgcgaggacacactgtgatcctgcacgcccagcccgggctccaggtgctgtgggatgggcagtcccaggtggaggtgagcgtacctggctcctaccagggccggacttgtgggctctgtgggaacttcaatggctttgcccaggacgatctgcagggccctgaggggctgctcctgccctcggaggctgcgtttgggaatagctggcaggtctcagaggggctgtggcctggccggccctgttctgcaggccgagaggtggatccgtgccgggcagcaggttaccgtgccaggcgtgaggccaatgcccggtgtggggtgctgaagtcctccccattcagtcgctgccatgctgtggtgccaccggagcccttctttgccgcctgtgtgtatgacctgtgtgcctgtggccctggctcctccgctgatgcctgcctctgtgatgccctggaagcctacgccagtcactgtcgccaggcaggagtgacacctacctggcgaggccccacgctgtgtgtggtaggctgccccctggagcgtggcttcgtgtttgatgagtgcggcccaccctgtccccgcacctgcttcaatcagcatatccccctgggggagctggcagcccactgcgtgaggccctgcgtgcccggctgccagtgccctgcaggcctggtggagcatgaggcccactgcatcccacccgaggcctgcccccaagtcctgctcactggagaccagccacttggtgctcggcccagccccagccgggagccccaggagacaccctgagccaggacagtgcctgataagggttcatcaggccaggagtctccccttggcgagcagttcccaccctggttagggctatggagagaatgccctgcctggacactggagcctgggcccctgccctgcaaagacccccgccatgttgagtcaccagcagtaaactctaggcctgcccgaa

SEQ ID NO: 5 - protein, 814 aa, Homo sapiens
MAGVGAAALSLLLHLGALALAAGAEGGAVPREPPGQQTTAHSSVLAGNSQEQWHPLREWLGRLEAAVMELREQNKDLQTRVRQLESCECHPASPQCWGLGRAWPEGARWEPDACTACVCQDGAAHCGPQAHLPHCRGCSQNGQTYGNGETFSPDACTTCRCLEGTITCNQKPCPRGPCPEPGACCPHCKPGCDYEGQLYEEGVTFLSSSNPCLQCTCLRSRVRCMALKCPPSPCPEPVLRPGHCCPTCQGCTEGGSHWEHGQEWTTPGDPCRICRCLEGHIQCRQRECASLCPYPARPLPGTCCPVCDGCFLNGREHRSGEPVGSGDPCSHCRCANGSVQCEPLPCPPVPCRHPGKIPGQCCPVCDGCEYQGHQYQSQETFRLQERGLCVRCSCQAGEVSCEEQECPVTPCALPASGRQLCPACELDGEEFAEGVQWEPDGRPCTACVCQDGVPKCGAVLCPPAPCQHPTQPPGACCPSCDSCTYHSQVYANGQNFTDADSPCHACHCQDGTVTCSLVDCPPTTCARPQSGPGQCCPRCPDCILEEEVFVDGESFSHPRDPCQECRCQEGHAHCQPRPCPRAPCAHPLPGTCCPNDCSGCAFGGKEYPSGADFPHPSDPCRLCRCLSGNVQCLARRCVPLPCPEPVLLPGECCPQCPAAPAPAGCPRPGAAHARHQEYFSPPGDPCRRCLCLDGSVSCQRLPCPPAPCAHPRQGPCCPSCDGCLYQGKEFASGERFPSPTAACHLCLCWEGSVSCEPKACAPALCPFPARGDCCPDCDGEGHGIGSCRGGMRETRGLGQNNLYCPRVDLKYLLQ

SEQ ID NO: 6 - DNA, 2774 bp, Homo sapiens
gagccgcgacgacagacggcgagccgagcgaggcggagctagcatggccggggtcggggccgctgcgctgtcccttctcctgcacctcggggccctggcgctggccgcgggcgcggaaggtggggctgtccccagggagccccctgggcagcagacaactgcccattcctcagtccttgctgggaactcccaggagcagtggcaccccctgcgagagtggctggggcgactggaggctgcagtgatggagctcagagaacagaataaggacctgcagacgagggtgaggcagctggagtcctgtgagtgccaccctgcatctccccagtgctgggggctggggcgtgcctggcccgagggggcacgctgggagcctgacgcctgcacagcctgcgtctgccaggatggggccgctcactgtggcccccaagcacacctgccccattgcaggggctgcagccaaaatggccagacctacggcaacggggagaccttctccccagatgcctgcaccacctgccgctgtctggaaggtaccatcacttgcaaccagaagccatgcccaagaggaccctgccctgagccaggagcatgctgcccgcactgtaagccaggctgtgattatgaggggcagctttatgaggagggggtcaccttcctgtccagctccaacccttgtctacagtgcacctgcctgaggagccgagttcgctgcatggccctgaagtgcccgcctagcccctgcccagagccagtgctgaggcctgggcactgctgcccaacctgccaaggctgcacagaaggtggctctcactgggaacatggccaagagtggacaacacctggggacccctgccgaatctgccggtgcctggagggtcacatccagtgccgccagcgagaatgtgccagcctgtgtccatacccagcccggcccctcccaggcacctgctgccctgtgtgtgatggctgtttcctaaacgggcgggagcaccgcagcggggagcctgtgggctcaggggacccctgctcgcactgccgctgtgctaatgggagtgtccagtgtgagcctctgccctgcccgccagtgccctgcagacacccaggcaagatccctgggcagtgctgccctgtctgcgatggctgtgagtaccagggacaccagtatcagagccaggagaccttcagactccaagagcggggcctctgtgtccgctgctcctgccaggctggcgaggtctcctgtgaggagcaggagtgcccagtcaccccctgtgccctgcctgcctctggccgccagctctgcccagcctgtgagctggatggagaggagtttgctgagggagtccagtgggagcctgatggtcggccctgcaccgcctgcgtctgtcaagatggggtacccaagtgcggggctgtgctctgccccccagccccctgccagcaccccacccagccccctggtgcctgctgccccagctgtgacagctgcacctaccacagccaagtgtatgccaatgggcagaacttcacggatgcagacagcccttgccatgcctgccactgtcaggatggaactgtgacatgctccttggttgactgccctcccacgacctgtgccaggccccagagtggaccaggccagtgttgccccaggtgcccagactgcatcctggaggaagaggtgtttgtggacggcgagagcttctcccacccccgagacccctgccaggagtgccgatgccaggaaggccatgcccactgccagcctcgcccctgccccagggccccctgtgcccacccgctgcctgggacctgctgcccgaacgactgcagcggctgtgcctttggcgggaaagagtaccccagcggagcggacttcccccacccctctgacccctgccgtctgtgtcgctgtctgagcggcaacgtgcagtgcctggcccgccgctgcgtgccgctgccctgtccagagcctgtcctgctgccgggagagtgctgcccgcagtgcccagccgccccagcccccgccggctgcccacggcccggcgcggcccacgcccgccaccaggagtacttctccccgcccggcgatccctgccgccgctgcctctgcctcgacggctccgtgtcctgccagcggctgccctgcccgcccgcgccctgcgcgcacccgcgccaggggccttgctgcccctcctgcgacggctgcctgtaccaggggaaggagtttgccagcggggagcgcttcccatcgcccactgctgcctgccacctctgcctttgctgggagggcagcgtgagctgcgagcccaaggcatgtgcccctgcactgtgccccttccctgccaggggcgactgctgccctgactgtgatggtgagggtcatgggatagggagctgccggggtgggatgcgggagaccagagggctgggtcagaataatctttactgccctagggtggatctaaaatatttattacagtaagaaaaagccccgaggctgggagccctagctgaagcctgtgaccccgacaatttgggaggctgaggcaggaggatcacttgagcccaggagttcaagaccagcctgggcaacatagagagatcttgtctctacacaaaaaatttaaaatcagctggtcgtggtgcctcttgtagttccatctactccggaggctgaggtgggaggattgcccaggagtttgaggctacagtgaaccgtgttttcaccactgcactccaggctgggtgacagagtgagaccttgtctc

(19) United States
(12) Patent Application Publication
(10) Pub. No.: US 2012/0202741 A1
(43) Pub. Date: Aug. 9, 2012

(54) DELIVERY OF BMP-7 AND METHODS OF USE THEREOF

(75) Inventors: Daniele Zink, Singapore (SG); Jackie Y. Ying, Singapore (SG); Edwin Pei Yong Chow, Singapore (SG); Farah Tasnim, Singapore (SG)

(73) Assignee: Agency for Science, Technology and Research, Connexis (SG)

(21) Appl. No.: 13/500,563

(22) PCT Filed: Oct. 6, 2010
(86) PCT No.: PCT/SG10/00380

§ 371 Date: Apr. 5, 2012

(30) Foreign Application Priority Data
Oct. 6, 2009 (SG) 200906666-3

(51) Int. Cl. A61K 38/18; C12M 3/00; A61M 1/14; C12M 3/06; B01D 63/02; B01D 61/24; C12N 5/071; C12N 5/10

(52) U.S. Cl. 514/8.8; 435/375; 435/377; 435/289.1; 435/297.1; 210/321.6; 210/198.1; 422/44

(57) ABSTRACT

The present invention generally relates to delivery of BMP-7 or functional variants or functional fragments thereof and/or a BMP-7 agonist and methods of use thereof. In some embodiments, methods and devices are provided for delivery of BMP-7 or functional variants or functional fragments thereof and/or a BMP-7 agonist to a patient. In some cases, the BMP-7 or functional variants or functional fragments thereof and/or a BMP-7 agonist may be released in controlled fashion from a device in fluid communication with a patient. In some embodiments, the BMP-7 or functional variants or functional fragments thereof and/or a BMP-7 agonist may be expressed by cells within a device. In other embodiments, methods are provided for improving the function of devices containing renal proximal tubule cells. For example, in some embodiments, exposure of renal proximal tubule cells to BMP-7 or functional variants or functional fragments thereof and/or a BMP-7 agonist may be used to inhibit disruption of cell layers comprising renal proximal tubule cells. In another embodiment, exposure of renal proximal tubule cells to BMP-7 or functional variants or functional fragments thereof and/or a BMP-7 agonist may be used to inhibit trans- and de-differentiation of renal proximal tubule cells. In another embodiment, exposure of renal proximal tubule cells to BMP-7 or functional variants or functional fragments thereof and/or a BMP-7 agonist may be used to improve renal proximal tubule cell functions.

### FIELD OF INVENTION

The present invention generally relates to delivery of BMP-7 or functional variants or functional fragments thereof and/or a BMP-7 agonist and methods of use thereof.

### BACKGROUND

Bioartificial kidneys (BAKs) contain a synthetic hemofilter connected in series with a bioreactor cartridge containing porous membranes, onto which renal proximal tubule cells are seeded. Results obtained with animal models of acute renal failure have shown that treatment with BAKs can improve cardiovascular performance, the levels of inflammatory cytokines, and survival time. A Phase II clinical trial revealed that BAK treatment improved survival of critically ill patients with acute renal failure as compared to conventional continuous renal replacement therapy.

Primary human renal proximal tubule cells (HPTCs) have been used for clinical applications of BAKs. Proximal tubule cells form a simple epithelium in vivo, and perform a variety of transport, metabolic, endocrinologic, and probably also immunomodulatory functions. Transport functions include the reabsorption of glucose, small solutes and bicarbonate from the glomerular filtrate, as well as the transport of toxins, xenobiotics, and drugs into the tubular lumen. In order to perform such functions efficiently in a BAK, HPTCs must form a well-differentiated epithelium with a controllable degree of leakiness on the porous membranes. However, spontaneous tubule formation on substrate surfaces (e.g., on or within tubular substrates) can lead to disruption of epithelia formed by HPTCs. Occurrence of such processes is problematic for BAK applications, where HPTCs are presented on porous membrane surfaces, and especially for hollow fiber BAKs.

### SUMMARY OF THE INVENTION

The present invention generally relates to delivery of BMP-7 or functional variants or functional fragments thereof and/or a BMP-7 agonist and methods of use thereof. The subject matter of the present invention involves, in some cases, interrelated products, alternative solutions to a particular problem, and/or a plurality of different uses of one or more systems and/or articles.

In one aspect, a method is provided. The method comprises contacting a plurality of renal proximal tubule cells in a fluidic device with sufficient BMP-7 or functional variants or functional fragments thereof and/or a BMP-7 agonist to inhibit tubule formation and/or improve cell performance by the plurality of renal proximal tubule cells.

In another aspect, a method is provided. The method comprises contacting a plurality of renal proximal tubule cells in a fluidic device with sufficient BMP-7 or functional variants or functional fragments thereof and/or a sufficient amount of a BMP-7 agonist to inhibit de-differentiation of the renal proximal tubule cells.

In still another aspect, a method is provided. The method comprises administering a therapeutic amount of BMP-7 or functional variants or functional fragments thereof and/or a BMP agonist systemically to a patient, wherein the BMP-7 or functional variants or functional fragments thereof and/or a BMP-7 agonist is generated essentially continuously from cells within a fluidic device comprising said cells in fluid communication with the patient.

In yet another aspect, a method is provided. The method comprises a fluidic device comprising a plurality of host cells genetically modified for overexpression of BMP-7 or functional variants or functional fragments thereof and/or a BMP-7 agonist.

In still another aspect, an apparatus is provided. The apparatus comprises a fluidic device comprising a semi-permeable membrane, wherein a non-cellular component of the apparatus is configured for controlled release of BMP-7 or functional variants or functional fragments thereof and/or a BMP-7 agonist.

In yet another aspect, a method is provided. The method comprises administering BMP-7 or functional variants or functional fragments thereof and/or a BMP-7 agonist systemically to a patient, wherein the BMP-7 is released from in controlled fashion from a non-cellular component within a fluidic device.

In still another aspect, a semi-permeable membrane is provided. The semi-permeable membrane comprises at least one material configured for controlled release of BMP-7 or functional variants or functional fragments thereof and/or a BMP-7 agonist.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a graph of hormone response assay results for parathyroid hormone and parathyroid hormone plus BMP-7, according to an embodiment;

FIG. 2 shows a graph of functional assay results for gamma-glutamyl transferase activity, according to an embodiment;

FIG. 3 shows a schematic of a hollow fiber bioartificial kidney, according to an embodiment;

FIG. 4 shows images of the formation and disruption of epithelia formed by HPTCs, according to an embodiment;

FIG. 5 shows images of the effects of different concentrations of BMP-7 and BMP-2, according to an embodiment;

FIG. 6 shows images of cells treated with BMP-7, according to an embodiment;

FIG. 7 shows a graph quantifying α-SMA/α-tubulin expression ratio at different concentrations of BMP-7 and BMP-2, according to an embodiment; and

FIG. 8 shows a graph comparing the amount of BMP-7 produced by HPTCs as a function of time.

### BRIEF DESCRIPTION OF THE SEQUENCES

SEQ ID NO. 1 is human bone morphogenetic protein-7 (BMP-7) having the amino acid sequence:

MHVRSLRAAAPHSFVALWAPLFLLRSALADFSLDNEVHSSFIHRRLRSQ
ERREMQREILSILGLPHRPRPHLQGKHNSAPMFMLDLYNAMAVEEGGGP
GGQGFSYPYKAVFSTQGPPLASLQDSHELTDADMVMSFVNLVEHDKEFF
HPRYHHREFRFDLSKIPEGEAVTAAEFRIYKDYIRERFDNETFRISVYQ
VLQEHLGRESDLFLLDSRTLWASEEGWLVFDITATSNHWVVNPRHNLGL
QLSVETLDGQSINPKLAGLIGRHGPQNKQPFMVAFFKATEVHFRSIRST
GSKQRSQNRSKTPKNQEALRMANVAENSSSDQRQACKKHELYVSFRDLG
WQDWIIAPEGYAAYYCEGECAFPLNSYMNATNHAIVQTLVHFINPETVP
KPCCAPTQLNAISVLYFDDSSNVILKKYRNMVVRACGCH;

SEQ ID NO. 2 is a cDNA sequence coding for human bone morphogenetic protein-7 (BMP-7) having the nucleic acid sequence:

ATGCACGTGCGCTCACTGCGAGCTGCGGCGCCGCACAGCTTCGTGGCGC
TCTGGGCACCCCTGTTCCTGCTGCGCTCCGCCCTGGCCGACTTCAGCCT
GGACAACGAGGTGCACTCGAGCTTCATCCACCGGCGCCTCCGCAGCCAG
GAGCGGCGGGAGATGCAGCGCGAGATCCTCTCCATTTTGGGCTTGCCCC
ACCGCCCGCGCCCGCACCTCCAGGGCAAGCACAACTCGGCACCCATGTT
CATGCTGGACCTGTACAACGCCATGGCGGTGGAGGAGGGCGGCGGGCCC
GGCGGCCAGGGCTTCTCCTACCCCTACAAGGCCGTCTTCAGTACCCAGG
GCCCCCCTCTGGCCAGCCTGCAAGATAGCCATTTCCTCACCGACGCCGA
CATGGTCATGAGCTTCGTCAACCTCGTGGAACATGACAAGGAATTCTTC
CACCCACGCTACCACCATCGAGAGTTCCGGTTTGATCTTTCCAAGATCC
CAGAAGGGGAAGCTGTCACGGCAGCCGAATTCCGGATCTACAAGGACTA
CATCCGGGAACGCTTCGACAATGAGACGTTCCGGATCAGCGTTTATCAG
GTGCTCCAGGAGCACTTGGGCAGGGAATCGGATCTCTTCCTGCTCGACA
GCCGTACCCTCTGGGCCTCGGAGGAGGGCTGGCTGGTGTTTGACATCAC
AGCCACCAGCAACCACTGGGTGGTCAATCCGCGGCACAACCTGGGCCTG
CAGCTCTCGGTGGAGACGCTGGATGGGCAGAGCATCAACCCCAAGTTGG
CGGGCCTGATTGGGCGGCACGGGCCCCAGAACAAGCAGCCCTTCATGGT
GGCTTTCTTCAAGGCCACGGAGGTCCACTTCCGCAGCATCCGGTCCACG
GGGAGCAAACAGCGCAGCCAGAACCGCTCCAAGACGCCCAAGAACCAGG
AAGCCCTGCGGATGGCCAACGTGGCAGAGAACAGCAGCAGCGACCAGAG
GCAGGCCTGTAAGAAGCACGAGCTGTATGTCAGCTTCCGAGACCTGGGC
TGGCAGGACTGGATCATCGCGCCTGAAGGCTACGCCGCCTACTACTGTG
AGGGGGAGTGTGCCTTCCCTCTGAACTCCTACATGAACGCCACCAACCA
CGCCATCGTGCAGACGCTGGTCCACTTCATCAACCCGGAAACGGTGCCC
AAGCCCTGCTGTGCGCCCACGCAGCTCAATGCCATCTCCGTCCTCTACT
TCGATGACAGCTCCAACGTCATCCTGAAGAAATACAGAAACATGGTGGT
CCGGGCCTGTGGCTGCCACTAG;

SEQ ID NO. 3 is human kielin/chordin-like protein (KCP) isoform 1 having the amino acid sequence:

MAGVGAAALSLLLHLGALALAAGAEGGAVPREPPGQQTTAHSSVLAGNS
QEQWHPLREWLGRLEAAVMELREQNKDLQTRVRQLESCECHPASPQCWG
LGRAWPEGARWEPDACTACVCQDGAAHCGPQAHLPHCRGCSQNGQTYGN
GETFSPDACTTCRCLTGAVQCQGPSCSELNCLESCTPPGECCPICCTEG
GSHWEHGQEWTTPGDPCRICRCLEGHIQCRQRECASLCPYPARPLPGTC
CPVCDGCFLNGREHRSGEPVGSGDPCSHCRCANGSVQCEPLPCPPVPCR
HPGKIPGQCCPVCDGCEYQGHQYQSQETFRLQERGLCVRCSCQAGEVSC
EEQECPVTPCALPASGRQLCPACELDGEEFAEGVQWEPDGRPCTACVCQ
DGVPKCGAVLCPPAPCQHPTQPPGACCPSCDSCTYHSQVYANGQNFTDA
DSPCHACHCQDGTVTCSLVDCPPTTCARPQSGPGQCCPRCPDCILEEEV
FVDGESFSHPRDPCQECRCQEGHAHCQPRPCPRAPCAHPLPGTCCPNDC
SGCAFGGKEYPSGADFPHPSDPCRLCRCLSGNVQCLARRCVPLPCPEPV
LLPGECCPQCPAPAGCPRPGAAHARHQEYFSPPGDPCRRCLCLDGSVSC
QRLPCPPAPCAHPRQGPCCPSCDGCLYQGKEFASGERFPSPTAACHLCL
CWEGSVSCEPKACAPALCPFPARGDCCPDCDGCEYLGESYLSNQEFPDP
REPCNLCTCLGGFVTCGRRPCEPPGCSHPLIPSGHCCPTCQGCRYHGVT
TASGETLPDPLDPTCSLCTCQEGSMRCQKKPCPPALCPHPSPGPCFCPV
CHSCLSQGREHQDGEEFEGPAGSCEWCRCQAGQVSCVRLQCPPLPCKLQ
VTERGSCCPRCRGCLAHGEEHPEGSRWVPPDSACSSCVCHEGVVTCARI
QCISSCAQPRQGPHDCCPQCSDCEHEGRKYEPGESFQPGADPCEVCICE
PQPEGPPSLRCHRRQCPSLVGCPPSQLLPPGPQHCCPTCAEALSNCSEG
LLGSELAPPDPCYTCQCQDLTWLCIHQACPELSCPLSERHTPPGSCCPV
CRAPTQSCVHQGREVASGERWTVDTCTSCSCMAGTVRCQSQRCSPLSCG
PDKAPALSPGSCCPRCLPRPASCMAFGDPHYRTFDGRLLHFQGSCSYVL
AKDCHSGDFSVHVTNDDRGRSGVAWTQEVAVLLGDMAVRLLQDGAVTVD
GHPVALPFLQEPLLYVELRGHTVILHAQPGLQVLWDGQSQVEVSVPGSY
QGRTCGLCGNFNGFAQDDLQGPEGLLLPSEAAFGNSWQVSEGLWPGRPC
SAGREVDPCRAAGYRARREANARCGVLKSSPFSRCHAVVPPEPFFAACV
YDLCACGPGSSADACLCDALEAYASHCRQAGVTPTWRGPTLCVVGCPLE
RGFVFDECGPPCPRTCFNQHIPLGELAAHCVRPCVPGCQCPAGLVEHEA
HCIPPEACPQVLLTGDQPLGARPSPSREPQETP;

SEQ ID NO. 4 is a cDNA sequence coding for human kielin/chordin-like protein (KCP), isoform 1 having the nucleic acid sequence:

GAGCCGCGACGACAGACGGCGAGCCGAGCGAGGCGGAGCTAGCATGGCC
GGGGTCGGGGCCGCTGCGCTGTCCCTTCTCCTGCACCTCGGGGCCCTGG
CGCTGGCCGCGGGCGCGGAAGGTGGGGCTGTCCCCAGGGAGCCCCCTGG
GCAGCAGACAACTGCCCATTCCTCAGTCCTTGCTGGGAACTCCCAGGAG
CAGTGGCACCCCCTGCGAGAGTGGCTGGGGCGACTGGAGGCTGCAGTGA
TGGAGCTCAGAGAACAGAATAAGGACCTGCAGACGAGGGTGAGGCAGCT
GGAGTCCTGTGAGTGCCACCCTGCATCTCCCCAGTGCTGGGGGCTGGGG
CGTGCCTGGCCCGAGGGGGCACGCTGGGAGCCTGACGCCTGCACAGCCT
GCGTCTGCCAGGATGGGGCCGCTCACTGTGGCCCCCAAGCACACCTGCC
CCATTGCAGGGGCTGCAGCCAAAATGGCCAGACCTACGGCAACGGGGAG
ACCTTCTCCCCAGATGCCTGCACCACCTGCCGCTGTCTGACAGGAGCCG
TGCAGTGCCAGGGGCCCTCGTGTTCAGAGCTCAACTGCTTGGAGAGCTG
CACCCCACCTGGGGAGTGCTGCCCCATCTGCTGCACAGAAGGTGGCTCT
CACTGGGAACATGGCCAAGAGTGGACAACACCTGGGGACCCCTGCCGAA
TCTGCCGGTGCCTGGAGGGTCACATCCAGTGCCGCCAGCGAGAATGTGC
CAGCCTGTGTCCATACCCAGCCCGGCCCCTCCCAGGCACCTGCTGCCCT
GTGTGTGATGGCTGTTTCCTAAACGGGCGGGAGCACCGCAGCGGGGAGC
CTGTGGGCTCAGGGGACCCCTGCTCGCACTGCCGCTGTGCTAATGGGAG
TGTCCAGTGTGAGCCTCTGCCCTGCCCGCCAGTGCCCTGCAGACACCCA
GGCAAGATCCCTGGGCAGTGCTGCCCTGTCTGCGATGGCTGTGAGTACC
AGGGACACCAGTATCAGAGCCAGGAGACCTTCAGACTCCAAGAGCGGGG
CCTCTGTGTCCGCTGCTCCTGCCAGGCTGGCGAGGTCTCCTGTGAGGAG
CAGGAGTGCCCAGTCACCCCCTGTGCCCTGCCTGCCTCTGGCCGCCAGC
TCTGCCCAGCCTGTGAGCTGGATGGAGAGGAGTTTGCTGAGGGAGTCCA
GTGGGAGCCTGATGGTCGGCCCTGCACCGCCTGCGTCTGTCAAGATGGG
GTACCCAAGTGCGGGGCTGTGCTCTGCCCCCCAGCCCCCTGCCAGCACC
CCACCCAGCCCCCTGGTGCCTGCTGCCCCAGCTGTGACAGCTGCACCTA
CCACAGCCAAGTGTATGCCAATGGGCAGAACTTCACGGATGCAGACAGC
CCTTGCCATGCCTGCCACTGTCAGGATGGAACTGTGACATGCTCCTTGG
TTGACTGCCCTCCCACGACCTGTGCCAGGCCCCAGAGTGGACCAGGCCA
GTGTTGCCCCAGGTGCCCAGACTGCATCCTGGAGGAAGAGGTGTTTGTG
GACGGCGAGAGCTTCTCCCACCCCCGAGACCCCTGCCAGGAGTGCCGAT
GCCAGGAAGGCCATGCCCACTGCCAGCCTCGCCCCTGCCCCAGGGCCCC
CTGTGCCCACCCGCTGCCTGGGACCTGCTGCCCGAACGACTGCAGCGGC
TGTGCCTTTGGCGGGAAAGAGTACCCCAGCGGAGCGGACTTCCCCCACC
CCTCTGACCCCTGCCGTCTGTGTCGCTGTCTGAGCGGCAACGTGCAGTG
CCTGGCCCGCCGCTGCGTGCCGCTGCCCTGTCCAGAGCCTGTCCTGCTG
CCGGGAGAGTGCTGCCCGCAGTGCCCAGCCCCCGCCGGCTGCCCACGGC
CCGGCGCGGCCCACGCCCGCCACCAGGAGTACTTCTCCCCGCCCGGCGA
TCCCTGCCGCCGCTGCCTCTGCCTCGACGGCTCCGTGTCCTGCCAGCGG
CTGCCCTGCCCGCCCGCGCCCTGCGCGCACCCGCGCCAGGGGCCTTGCT
GCCCCTCCTGCGACGGCTGCCTGTACCAGGGGAAGGAGTTTGCCAGCGG
GGAGCGCTTCCCATCGCCCACTGCTGCCTGCCACCTCTGCCTTTGCTGG
GAGGGCAGCGTGAGCTGCGAGCCCAAGGCATGTGCCCCTGCACTGTGCC
CCTTCCCTGCCAGGGGCGACTGCTGCCCTGACTGTGATGGCTGTGAGTA
CCTGGGGGAGTCCTACCTGAGTAACCAGGAGTTCCCAGACCCCCGAGAA
CCCTGCAACCTGTGTACCTGTCTTGGAGGCTTCGTGACCTGCGGCCGCC
GGCCCTGTGAGCCTCCGGGCTGCAGCCACCCACTCATCCCCTCTGGGCA
CTGCTGCCCGACCTGCCAGGGATGCCGCTACCATGGCGTCACTACTGCC
TCCGGAGAGACCCTTCCTGACCCACTTGACCCTACCTGCTCCCTCTGCA
CCTGCCAGGAAGGTTCCATGCGCTGCCAGAAGAAGCCATGTCCCCCAGC
TCTCTGCCCCCACCCCTCTCCAGGCCCCTGCTTCTGCCCTGTTTGCCAC
AGCTGTCTCTCTCAGGGCCGGGAGCACCAGGATGGGGAGGAGTTTGAGG
GACCAGCAGGCAGCTGTGAGTGGTGTCGCTGTCAGGCTGGCCAGGTCAG
CTGTGTGCGGCTGCAGTGCCCACCCCTTCCCTGCAAGCTCCAGGTCACC
GAGCGGGGGAGCTGCTGCCCTCGCTGCAGAGGCTGCCTGGCTCATGGGG
AAGAGCACCCCGAAGGCAGTAGATGGGTGCCCCCCGACAGTGCCTGCTC
CTCCTGTGTGTGTCACGAGGGCGTCGTCACCTGTGCACGCATCCAGTGC
ATCAGCTCTTGCGCCCAGCCCCGCCAAGGGCCCCATGACTGCTGTCCTC
AATGCTCTGACTGTGAGCATGAGGGCCGGAAGTACGAGCCTGGGGAGAG
CTTCCAGCCTGGGGCAGACCCCTGTGAAGTGTGCATCTGCGAGCCACAG
CCTGAGGGGCCTCCCAGCCTTCGCTGTCACCGGCGGCAGTGTCCCAGCC
TGGTGGGCTGCCCCCCCAGCCAGCTCCTGCCCCCTGGGCCCCAGCACTG
CTGTCCCACCTGTGCCGAGGCCTTGAGTAACTGTTCAGAGGGCCTGCTG
GGATCTGAGCTAGCCCCACCAGACCCCTGCTACACGTGCCAGTGCCAGG
ACCTGACATGGCTCTGCATCCACCAGGCTTGTCCTGAGCTCAGCTGTCC
CCTCTCAGAGCGCCACACTCCCCCTGGGAGCTGCTGCCCCGTATGCCGG
GCTCCCACCCAGTCCTGCGTGCACCAGGGCCGTGAGGTGGCCTCTGGAG
AGCGCTGGACTGTGGACACCTGCACCAGCTGCTCCTGCATGGCGGGCAC
CGTGCGTTGCCAGAGCCAGCGCTGCTCACCGCTCTCGTGTGGCCCCGAC
AAGGCCCCTGCCCTGAGTCCTGGCAGCTGCTGCCCCCGCTGCCTGCCTC
GGCCCGCTTCCTGCATGGCCTTCGGAGACCCCCATTACCGCACCTTCGA
CGGCCGCCTGCTGCACTTCCAGGGCAGTTGCAGCTATGTGCTGGCCAAG
GACTGCCACAGCGGGGACTTCAGTGTGCACGTGACCAATGATGACCGGG
GCCGGAGCGGTGTGGCCTGGACCCAGGAGGTGGCGGTGCTGCTGGGAGA
CATGGCCGTGCGGCTGCTGCAGGACGGGGCAGTCACGGTGGATGGGCAC
CCGGTGGCCTTGCCCTTCCTGCAGGAGCCGCTGCTGTATGTGGAGCTGC
GAGGACACACTGTGATCCTGCACGCCCAGCCCGGGCTCCAGGTGCTGTG
GGATGGGCAGTCCCAGGTGGAGGTGAGCGTACCTGGCTCCTACCAGGGC
CGGACTTGTGGGCTCTGTGGGAACTTCAATGGCTTTGCCCAGGACGATC
TGCAGGGCCCTGAGGGGCTGCTCCTGCCCTCGGAGGCTGCGTTTGGGAA
TAGCTGGCAGGTCTCAGAGGGGCTGTGGCCTGGCCGGCCCTGTTCTGCA
GGCCGAGAGGTGGATCCGTGCCGGGCAGCAGGTTACCGTGCCAGGCGTG
AGGCCAATGCCCGGTGTGGGGTGCTGAAGTCCTCCCCATTCAGTCGCTG
CCATGCTGTGGTGCCACCGGAGCCCTTCTTTGCCGCCTGTGTGTATGAC
CTGTGTGCCTGTGGCCCTGGCTCCTCCGCTGATGCCTGCCTCTGTGATG
CCCTGGAAGCCTACGCCAGTCACTGTCGCCAGGCAGGAGTGACACCTAC
CTGGCGAGGCCCCACGCTGTGTGTGGTAGGCTGCCCCCTGGAGCGTGGC
TTCGTGTTTGATGAGTGCGGCCCACCCTGTCCCCGCACCTGCTTCAATC
AGCATATCCCCCTGGGGGAGCTGGCAGCCCACTGCGTGAGGCCCTGCGT
GCCCGGCTGCCAGTGCCCTGCAGGCCTGGTGGAGCATGAGGCCCACTGC
ATCCCACCCGAGGCCTGCCCCCAAGTCCTGCTCACTGGAGACCAGCCAC
TTGGTGCTCGGCCCAGCCCCAGCCGGGAGCCCCAGGAGACACCCTGAGC
CAGGACAGTGCCTGATAAGGGTTCATCAGGCCAGGAGTCTCCCCTTGGC
GAGCAGTTCCCACCCTGGTTAGGGCTATGGAGAGAATGCCCTGCCTGGA
CACTGGAGCCTGGGCCCCTGCCCTGCAAAGACCCCCGCCATGTTGAGTC
ACCAGCAGTAAACTCTAGGCCTGCCCGAA;

SEQ ID NO. 5 is human kielin/chordin-like protein (KCP) isoform 2 having the amino acid sequence:

MAGVGAAALSLLLHLGALALAAGAEGGAVPREPPGQQTTAHSSVLAGNS
QEQWHPLREWLGRLEAAVMELREQNKDLQTRVRQLESCECHPASPQCWG
LGRAWPEGARWEPDACTACVCQDGAAHCGPQAHLPHCRGCSQNGQTYGN
GETFSPDACTTCRCLEGTITCNQKPCPRGPCPEPGACCPHCKPGCDYEG
QLYEEGVTFLSSSNPCLQCTCLRSRVRCMALKCPPSPCPEPVLRPGHCC
PTCQGCTEGGSHWEHGQEWTTPGDPCRICRCLEGHIQCRQRECASLCPY
PARPLPGTCCPVCDGCFLNGREHRSGEPVGSGDPCSHCRCANGSVQCEP
LPCPPVPCRHPGKIPGQCCPVCDGCEYQGHQYQSQETFRLQERGLCVRC
SCQAGEVSCEEQECPVTPCALPASGRQLCPACELDGEEFAEGVQWEPDG
RPCTACVCQDGVPKCGAVLCPPAPCQHPTQPPGACCPSCDSCTYHSQVY
ANGQNFTDADSPCHACHCQDGTVTCSLVDCPPTTCARPQSGPGQCCPRC
PDCILEEEVFVDGESFSHPRDPCQECRCQEGHAHCQPRPCPRAPCAHPL
PGTCCPNDCSGCAFGGKEYPSGADFPHPSDPCRLCRCLSGNVQCLARRC
VPLPCPEPVLLPGECCPQCPAAPAPAGCPRPGAAHARHQEYFSPPGDPC
RRCLCLDGSVSCQRLPCPPAPCAHPRQGPCCPSCDGCLYQGKEFASGER
FPSPTAACHLCLCWEGSVSCEPKACAPALCPFPARGDCCPDCDGEGHGI
GSCRGGMRETRGLGQNNLYCPRVDLKYLLQ;

and

SEQ ID NO. 6 is a cDNA sequence coding for human kielin/chordin-like protein (KCP), isoform 2 having the nucleic acid sequence:

GAGCCGCGACGACAGACGGCGAGCCGAGCGAGGCGGAGCTAGCATGGCC
GGGGTCGGGGCCGCTGCGCTGTCCCTTCTCCTGCACCTCGGGGCCCTGG
CGCTGGCCGCGGGCGCGGAAGGTGGGGCTGTCCCCAGGGAGCCCCCTGG
GCAGCAGACAACTGCCCATTCCTCAGTCCTTGCTGGGAACTCCCAGGAG
CAGTGGCACCCCCTGCGAGAGTGGCTGGGGCGACTGGAGGCTGCAGTGA
TGGAGCTCAGAGAACAGAATAAGGACCTGCAGACGAGGGTGAGGCAGCT
GGAGTCCTGTGAGTGCCACCCTGCATCTCCCCAGTGCTGGGGGCTGGGG
CGTGCCTGGCCCGAGGGGGCACGCTGGGAGCCTGACGCCTGCACAGCCT
GCGTCTGCCAGGATGGGGCCGCTCACTGTGGCCCCCAAGCACACCTGCC
CCATTGCAGGGGCTGCAGCCAAAATGGCCAGACCTACGGCAACGGGGAG
ACCTTCTCCCCAGATGCCTGCACCACCTGCCGCTGTCTGGAAGGTACCA
TCACTTGCAACCAGAAGCCATGCCCAAGAGGACCCTGCCCTGAGCCAGG
AGCATGCTGCCCGCACTGTAAGCCAGGCTGTGATTATGAGGGGCAGCTT
TATGAGGAGGGGGTCACCTTCCTGTCCAGCTCCAACCCTTGTCTACAGT
GCACCTGCCTGAGGAGCCGAGTTCGCTGCATGGCCCTGAAGTGCCCGCC
TAGCCCCTGCCCAGAGCCAGTGCTGAGGCCTGGGCACTGCTGCCCAACC
TGCCAAGGCTGCACAGAAGGTGGCTCTCACTGGGAACATGGCCAAGAGT
GGACAACACCTGGGGACCCCTGCCGAATCTGCCGGTGCCTGGAGGGTCA
CATCCAGTGCCGCCAGCGAGAATGTGCCAGCCTGTGTCCATACCCAGCC
CGGCCCCTCCCAGGCACCTGCTGCCCTGTGTGTGATGGCTGTTTCCTAA
ACGGGCGGGAGCACCGCAGCGGGGAGCCTGTGGGCTCAGGGGACCCCTG
CTCGCACTGCCGCTGTGCTAATGGGAGTGTCCAGTGTGAGCCTCTGCCC
TGCCCGCCAGTGCCCTGCAGACACCCAGGCAAGATCCCTGGGCAGTGCT
GCCCTGTCTGCGATGGCTGTGAGTACCAGGGACACCAGTATCAGAGCCA
GGAGACCTTCAGACTCCAAGAGCGGGGCCTCTGTGTCCGCTGCTCCTGC
CAGGCTGGCGAGGTCTCCTGTGAGGAGCAGGAGTGCCCAGTCACCCCCT
GTGCCCTGCCTGCCTCTGGCCGCCAGCTCTGCCCAGCCTGTGAGCTGGA
TGGAGAGGAGTTTGCTGAGGGAGTCCAGTGGGAGCCTGATGGTCGGCCC
TGCACCGCCTGCGTCTGTCAAGATGGGGTACCCAAGTGCGGGGCTGTGC
TCTGCCCCCCAGCCCCCTGCCAGCACCCCACCCAGCCCCCTGGTGCCTG
CTGCCCCAGCTGTGACAGCTGCACCTACCACAGCCAAGTGTATGCCAAT
GGGCAGAACTTCACGGATGCAGACAGCCCTTGCCATGCCTGCCACTGTC
AGGATGGAACTGTGACATGCTCCTTGGTTGACTGCCCTCCCACGACCTG
TGCCAGGCCCCAGAGTGGACCAGGCCAGTGTTGCCCCAGGTGCCCAGAC
TGCATCCTGGAGGAAGAGGTGTTTGTGGACGGCGAGAGCTTCTCCCACC
CCCGAGACCCCTGCCAGGAGTGCCGATGCCAGGAAGGCCATGCCCACTG
CCAGCCTCGCCCCTGCCCCAGGGCCCCCTGTGCCCACCCGCTGCCTGGG
ACCTGCTGCCCGAACGACTGCAGCGGCTGTGCCTTTGGCGGGAAAGAGT
ACCCCAGCGGAGCGGACTTCCCCCACCCCTCTGACCCCTGCCGTCTGTG
TCGCTGTCTGAGCGGCAACGTGCAGTGCCTGGCCCGCCGCTGCGTGCCG
CTGCCCTGTCCAGAGCCTGTCCTGCTGCCGGGAGAGTGCTGCCCGCAGT
GCCCAGCCGCCCCAGCCCCCGCCGGCTGCCCACGGCCCGGCGCGGCCCA
CGCCCGCCACCAGGAGTACTTCTCCCCGCCCGGCGATCCCTGCCGCCGC
TGCCTCTGCCTCGACGGCTCCGTGTCCTGCCAGCGGCTGCCCTGCCCGC
CCGCGCCCTGCGCGCACCCGCGCCAGGGGCCTTGCTGCCCCTCCTGCGA
CGGCTGCCTGTACCAGGGGAAGGAGTTTGCCAGCGGGGAGCGCTTCCCA
TCGCCCACTGCTGCCTGCCACCTCTGCCTTTGCTGGGAGGGCAGCGTGA
GCTGCGAGCCCAAGGCATGTGCCCCTGCACTGTGCCCCTTCCCTGCCAG
GGGCGACTGCTGCCCTGACTGTGATGGTGAGGGTCATGGGATAGGGAGC
TGCCGGGGTGGGATGCGGGAGACCAGAGGGCTGGGTCAGAATAATCTTT
ACTGCCCTAGGGTGGATCTAAAATATTTATTACAGTAAGAAAAAGCCCC
GAGGCTGGGAGCCCTAGCTGAAGCCTGTGACCCCGACAATTTGGGAGGC
TGAGGCAGGAGGATCACTTGAGCCCAGGAGTTCAAGACCAGCCTGGGCA
ACATAGAGAGATCTTGTCTCTACACAAAAAATTTAAAATCAGCTGGTCG
TGGTGCCTCTTGTAGTTCCATCTACTCCGGAGGCTGAGGTGGGAGGATT
GCCCAGGAGTTTGAGGCTACAGTGAACCGTGTTTTCACCACTGCACTCC
AGGCTGGGTGACAGAGTGAGACCTTGTCTC.

### DETAILED DESCRIPTION

The present invention generally relates to delivery of BMP-7 or functional variants or functional fragments thereof and/or a BMP-7 agonist or functional variants or functional fragments thereof and methods of use thereof. In some embodiments, methods and devices are provided for delivery of BMP-7 or functional variants or functional fragments thereof and/or a BMP-7 agonist to a patient. In some cases, the BMP-7 or functional variants or functional fragments thereof and/or a BMP-7 agonist may be released in controlled fashion from a fluidic device, such as but not limited to, a BAK device, in fluid communication with a patient. In some embodiments, the BMP-7 or functional variants or functional fragments thereof and/or a BMP-7 agonist may be expressed by cells within a device or may be released in a controlled fashion by a non-cellular component within a device, as described in more detail below. In other embodiments, methods are provided for improving the function of devices containing renal proximal tubule cells. For example, in some embodiments, exposure of renal proximal tubule cells to BMP-7 or functional variants or functional fragments thereof and/or a BMP-7 agonist may be used to inhibit disruption of cell layers comprising renal proximal tubule cells. In another embodiment, exposure of renal proximal tubule cells to BMP-7 or functional variants or functional fragments thereof and/or a BMP-7 agonist may be used to inhibit trans- and de-differentiation of renal proximal tubule cells. In another embodiment, exposure of renal proximal tubule cells to BMP-7 or functional variants or functional fragments thereof and/or a BMP-7 agonist may be used to improve renal proximal tubule cell functions (e.g transport, metabolic and/or endocriniologic functions).

In some embodiments, renal proximal tubule cells may be used to form an epithelium on a membrane (i.e., the cells may reside on the membrane). In some embodiments, such a configuration may be useful, for example, in a device where it is desired that renal proximal tubule cells control the flow of fluid and transport of solute from a first region of the device to a second region of the device. For instance, in some embodiments, a membrane with a layer of renal proximal tubule cells may be used in a reabsorption unit of a bioartificial kidney or another unit of a cell-containing device, as described in more detail below.

In some cases, the renal proximal tubule cells may form a confluent layer on the membrane. As discussed in more detail below, the membrane may be semi-permeable in some embodiments. In some cases, it is desirable that the cell layer be essentially free of gaps, thereby preventing fluid from leaking around the cells. Additionally, in some embodiments, the renal proximal tubule cells should be capable of performing molecular transport functions (e.g., transporting glucose and other substances). Generally, renal proximal tubule cells should be differentiated to a point such that the cells are capable of performing the transport, metabolic and endocrinologic functions typical for renal proximal tubule cells. In some embodiments, the renal proximal tubule cells may be obtained from human subjects or other mammalian subjects.

In some embodiments, the renal proximal tubule cells can spontaneously form tubules when growing on a surface (e.g., a membrane), especially when the surface has a high amount of curvature, such as in the case of tubular structures. For example, in some embodiments, renal proximal tubule cells are more prone to form tubules spontaneously when seeded on a surface of a hollow fiber membrane. As a result, or in some cases for other reasons, the renal proximal tubule cell layer on the membrane can be disrupted. This can be deleterious, for example, since control of transport processes through the membrane may be reduced or eliminated. Additionally, renal proximal tubule cells may aggregate, which can also disrupt the cell layer on the membrane. Furthermore, myofibroblasts (i.e., myofibroblasts generated by trans-differentiation of renal proximal tubule cells) can accumulate on the membrane, which also can be disadvantageous since these cells do not provide renal proximal tubule cell functions. Without wishing to be bound by any theory, it is believed that in some cases, myofibroblasts can accumulate when renal proximal tubule cells undergo epithelial-to-mesenchymal transdifferentiation to form myofibroblasts. In some embodiments, cell aggregation and/or tubule formation can lead to clogging of fluidic devices (e.g., BAKs and/or other fluidic devices comprising renal proximal tubule cells). For example, cell aggregation and/or tubule formation by renal proximal tubule cells growing on the inside of tubular membranes (e.g., hollow fiber membranes) can cause clogging of the tubular membranes.

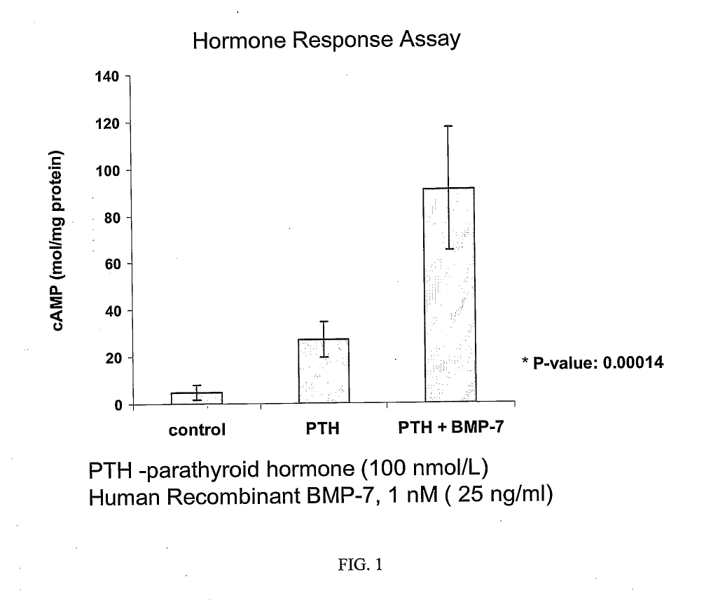

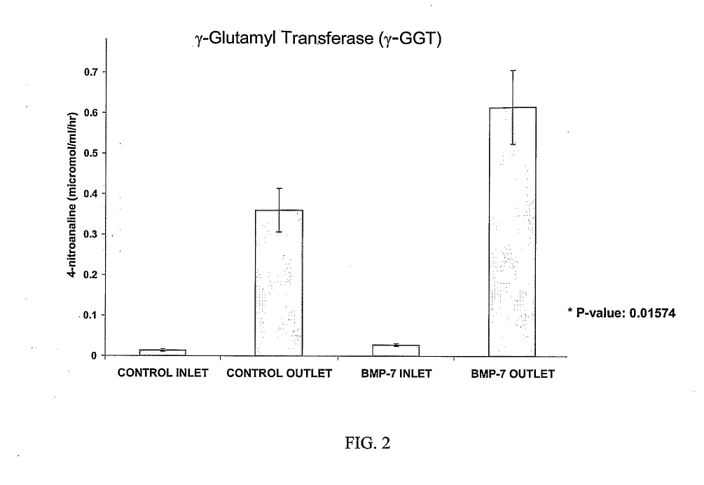

It has been surprisingly discovered that tubule formation, trans- and/or de-differentiation, and/or disruption of renal proximal tubule cell layers on membranes can be inhibited by exposing the cells to bone morphogenetic protein-7 (BMP-7) or functional variants or functional fragments thereof and/or a BMP-7 agonist. In some embodiments, BMP-7 or functional variants or functional fragments thereof and/or a BMP-7 agonist also may improve certain cellular functions. For example, BMP-7 or functional variants or functional fragments thereof and/or a BMP-7 agonist may improve the response of HPTCs to parathyroid hormone (FIG. 1). In another example, in some cases, BMP-7 or functional variants or functional fragments thereof and/or a BMP-7 agonist may improve gamma-glutamyltransferase (GGT) activity of HPTCs, as demonstrated in FIG. 2, which shows the gamma-glutamyltransferase activity in cell culture medium before entering a flat-bed bioreactor (inlet) and after passing through the flat-bed bioreactor (outlet).

BMP-7 is a member of the transforming growth factor (TGF)-β superfamily. It should be understood that BMP-7 refers to a human protein encoded by the amino acid sequence of SEQ ID NO. 1. In some embodiments, the amino acid sequence of BMP-7 is SEQ ID NO. 1. In certain embodiments, rather than using BMP-7, a functional variant or functional fragment thereof may be employed. In some embodiments, the amino acid sequence of BMP-7 may be coded for by the nucleic acid sequence of SEQ ID NO. 2. In certain embodiments, the amino acid sequence of BMP-7 may be coded for by the complement of a nucleic acid sequence that hybridizes to the nucleic acid sequence of SEQ ID NO. 2 under high stringency conditions. Such nucleic acids may be DNA, RNA, composed of mixed deoxyribonucleotides and ribonucleotides, or may also incorporate synthetic non-natural nucleotides. Various methods for determining the expression of a nucleic acid and/or a polypeptide in normal and tumor cells are known to those of skill in the art. In certain embodiments, a non-human ortholog of BMP-7 or functional variants or functional fragments thereof may be used.

The term “highly stringent conditions” or “high stringency conditions” as used herein refers to parameters with which those skilled in the art are familiar. Nucleic acid hybridization parameters may be found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, stringent conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH2PO4 (pH 7), 0.5% SDS, 2 mM EDTA). SSC is 0.15M sodium chloride/0.15M sodium citrate, pH 7; SDS is sodium dodecyl sulphate; and EDTA is ethylenediaminetetracetic acid. After hybridization, the membrane upon which the DNA is transferred is washed at 2×SSC at room temperature and then at 0.1×SSC/0.1×SDS at temperatures up to 68° C.

The foregoing set of hybridization conditions is but one example of highly stringent hybridization conditions known to one of ordinary skill in the art. There are other conditions, reagents, and so forth which can be used, which result in a highly stringent hybridization. The skilled artisan will be familiar with such conditions, and thus they are not given here. It will be understood, however, that the skilled artisan will be, able to manipulate the conditions in a manner to permit the clear identification of homologs and alleles of the nucleic acid molecules of the invention. The skilled artisan also is familiar with the methodology for screening cells and libraries for expression of such molecules which then are routinely isolated, followed by isolation of the pertinent nucleic acid molecule and sequencing.

The invention also includes use of degenerate nucleic acid molecules which include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating peptide sequence of the invention. Similarly, nucleotide sequence triplets which encode other amino acid residues include, but are not limited to: CCA, CCC, CCG and CCT (proline codons); CGA, CGC, CGG, CGT, AGA and AGG (arginine codons); ACA, ACC, ACG and ACT (threonine codons); AAC and AAT (asparagine codons); and ATA, ATC and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

“Functional variant” or “functional fragment” as those terms are used herein, is a protein that differs from a reference protein (i.e. a BMP-7 protein or fragment thereof, or an agonist or fragment thereof, consistent with embodiments of the present invention), but retains essential properties (i.e., biological activity). A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. Generally, differences are limited so that the sequences of the reference polypeptide and the variant or fragment are closely similar overall and, in many regions, identical.

A functional variant or functional fragment and reference protein may differ in amino acid sequence by one or more substitutions, additions, and deletions in any combination. A substituted or inserted amino acid residue may or may not be one encoded by the genetic code. A variant of a protein may be naturally occurring such as an allelic variant, or it may be a variant that is not known to occur naturally. Non-naturally occurring variants of polynucleotides and polypeptides may be made by mutagenesis techniques or by direct synthesis. For instance, a conservative amino acid substitution may be made with respect to the amino acid sequence encoding the polypeptide.

Functional variant or functional fragment proteins encompassed by the present application are biologically active, that is they continue to possess the desired biological activity of the native protein, as described herein. The term “functional variant” includes, but is not limited to, any polypeptide having an amino acid residue sequence substantially identical to a sequence specifically shown herein in which one or more residues have been conservatively substituted with a functionally similar residue, and which displays the ability to inhibit tubule formation by renal proximal tubule cells and/or de-differentiation of renal proximal tubule cells and/or which improves cellular functions. “Biological activity,” as used herein refers to the ability of the protein to inhibit tubule formation by renal proximal tubule cells, as assayed by histological examination (e.g. See Example 1), and/or to improve cell performance by renal proximal tubule cells. “Improve cell performance,” refers to a statistically significant increase in the level of GGT activity and responsiveness to parathyroid hormone as assayed by quantification of GGT activity and quantification of responsiveness to parathyroid hormone (e.g. see Example 5). A “statistically significant increase” refers to a p-value being less than a threshold level when comparing the assay results of treated and untreated cells. The p-value is calculated using an unpaired Student's t-test. In some embodiments, a statistically significant increase may refer to a p-value less than 0.10, in some embodiments less than 0.05, in some embodiments less than 0.01, in some embodiments less than 0.005, and in some embodiments less than 0.001. Functional variants may result from, for example, genetic polymorphism or from human manipulation. Biologically active variants and fragments (i.e. functional variants and functional fragments) of a BMP-7 protein of the invention will have at least about 75%, 80%, 85%, 90%, 91%, 92%, 93%, 94%, 95%, 96%, 97%, 98%, 99% or more sequence identity to the amino acid sequence for the human BMP-7 protein as determined by sequence alignment programs and parameters described elsewhere herein. A biologically active variant of a protein consistent with an embodiment of the invention may differ from that protein by as few as 1-15 amino acid residues, as few as 1-10, such as 6-10, as few as 5, as few as 4, 3, 2, or even 1 amino acid residue.

In some embodiments, a functional variant or fragment of SEQ ID NO. 1 typically will share with SEQ ID NO. 1 at least 75% amino acid identity, in some instances at least 80% amino acid identity, in some instances at least 90% amino acid identity, in some instances at least 95% amino acid identity, in some instances at least 96% amino acid identity, in some instances at least 97% amino acid identity, in some instances at least 98% amino acid identity, and in some instances at least 99% amino acid identity. The percent identity can be calculated using various, publicly available software tools developed by NCBI (Bethesda, Md.) that can be obtained through the internet (ftp:/ncbi.nlm.nih.gov/pub/). Exemplary tools include the BLAST system available at http://www.ncbi.nlm.nih.gov, which uses algorithms developed by Altschul et al. (Nucleic Acids Res. 25:3389-3402, 1997). Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acid molecules also are embraced by the invention.

It should be understood that BMP-7 may be modified, for example through mutation, chemical modification, truncation, fusion with another protein, etc. while still substantially retaining its therapeutic and/or functional ability, for example to inhibit aggregation and/or tubule formation by renal proximal tubule cells. Such modified products still comprise BMP-7, or functional variants or functional fragments thereof, as used herein. Other examples of modifications include posttranslational modifications; for example, BMP-7 as used herein also encompasses BMP-7 that may be glycosylated, acylated, methylated, phosphorylated, lipoylated, etc.

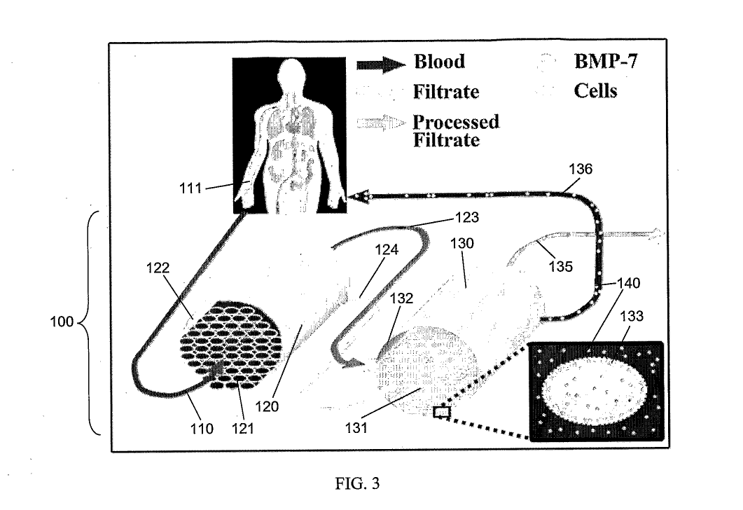

In some embodiments, the invention involves use of a fluidic device. Non-limiting examples of fluidic devices include BAKs, dialysis machines, and controlled release devices. In some embodiments, the devices include cells. For example, the devices may include renal proximal tubule cells and/or other cells, as described below. In some embodiments, a fluidic device may not incorporate cells. For instance, a fluidic device may not need cells to release BMP-7, or functional variants or functional fragments thereof, and/or a BMP-7 agonist for systemic uptake. For example, a controlled release device may release BMP-7, or functional variants or functional fragments thereof, and/or a BMP-7 agonist without the use of cells. In another example, a dialysis machine may perform blood filtering without the use of cells and may also be capable of releasing BMP-7, or functional variants or functional fragments thereof, and/or a BMP-7 agonist. In some embodiments, a BAK may be used that has a reabsorption unit that may utilize a hollow fiber membrane seeded with renal proximal tubule cells. Such embodiments have been described, for example, in Humes et al. Kidney International (1999), 55, 2502, and in Saito et al. J. Artificial Organs (2006) 9, 130, each of which is incorporated herein by reference. A non-limiting example of a BMP-7-delivering hollow fiber BAK is shown in FIG. 3. The BAK 100 comprises an inlet 110 that is in fluid communication with the circulation system 111 of a subject. Blood flows into the filtration unit 120 through the inlet. The filtration unit comprises a plurality of hollow fiber membranes 121 through which fluid, but not cells, can pass. “Permeate” refers to the fluid that has been passed through the membrane. “Retentate” refers to the portion of the blood that does not cross the membrane. The blood flows into the hollow fibers of the filtration unit and fluid from the blood passes through the hollow fiber membranes resulting in formation of a permeate in the spaces 122 exterior to the hollow fibers. The retentate 123 and permeate 124 then flow into the reabsorption unit 130. The reabsorption unit comprises hollow fiber membranes 131 into which the permeate from the filtration unit flows. The retentate from the filtration unit flows into the spaces 132 exterior to the hollow fibers. The interior surface of the hollow fibers of the reabsorption unit has renal proximal tubule cells 133 seeded thereon. The permeate from the filtration unit flows into hollow fibers of the reabsorption unit where it contacts the renal proximal tubule cells. A portion of the fluid from the permeate passes through the hollow fibers seeded with renal proximal tubule cells into the spaces exterior to the hollow fibers. This fluid is herein referred to as the “reabsorbate.” Like the tubules of the kidney, the human proximal tubule cells perform their biological functions in regulating the reabsorption and metabolism of important substances such as glucose, water and ions. In some non-limiting embodiments, BMP-7 140 may be released within the device, for example, from a component within the reabsorption unit or from cells within the reabsorption unit. The residual permeate 135 flows out of the BAK and into a waste container. In some embodiments, the combined retentate and reabsorbate 136, which are enriched in BMP-7, flows out of the BAK and back into the circulation system of a subject.

In some embodiments, a flat-bed BAK may be used, for example, as described in an International Patent Application, filed on Oct. 4, 2010, entitled, “Improved Bioartificial Kidneys,” by Ying et al., which is incorporated herein by reference.

In embodiments where a BAK is employed, BMP-7 or functional variants or functional fragments thereof and/or a BMP-7 agonist may be delivered to the renal proximal tubule cells on the membrane of such device in various ways. For example, in one embodiment, the renal proximal tubule cells may be cocultured with one or more cell types that express BMP-7 or functional variants or functional fragments thereof and/or a BMP-7 agonist. Generally, the one or more cell types that express BMP-7 or functional variants or functional fragments thereof and/or a BMP-7 agonist should be capable of expressing BMP-7 or functional variants or functional fragments thereof and/or a BMP-7 agonist in an amount sufficient to improve proximal tubule cell functions, inhibit tubule formation, trans- and/or de-differentiation, and/or disruption of the renal proximal tubule cell layer. In some embodiments, renal proximal tubule cells not expressing BMP-7 may be cocultured with distal tubule cells, collecting duct cells, podocytes, cells of the thick ascending limb, and/or other renal cell types that express BMP-7. In some embodiments, the renal proximal tubule cells may be cocultured with cells that express erythropoietin, for example, such as renal fibroblasts. In some embodiments, the amount of BMP-7 or functional variants or functional fragments thereof and/or a BMP-7 agonist produced by the cells on the membrane may be controlled by the ratio of renal proximal tubule cells to the one or more cell types expressing BMP-7 or functional variants or functional fragments thereof and/or a BMP-7 agonist. In some cases, the ratio of renal proximal tubule cells to the one or more cell types expressing BMP-7 or functional variants or functional fragments thereof and/or a BMP-7 agonist may be less than 1000:1, less than 100:1, less than 50:1, less than 20:1, less than 10:1, or less than 5:1. Alternatively, in some embodiments, cells expressing BMP-7 may not be cocultured with renal proximal tubule cells but, rather, may be located in a different region of a device and be in fluid communication with the renal proximal tubule cells. In some embodiments, cells that constitutively produce BMP-7 may be used in the absence of renal proximal tubule cells. For example, cells such as distal tubule cells, collecting duct cells, podocytes, cells of the thick ascending limb, and/or other renal cell types that express BMP-7 be used in the absence of renal proximal tubule cells. In some embodiments, cells that express erythropoietin, for example, such as renal fibroblasts, may be used in the absence of renal proximal tubule cells.

In another aspect of the invention, a nucleotide sequence such as one encoding BMP-7 is delivered into renal proximal tubule cells and/or other cell types. Any method or delivery system may be used for the delivery and/or transfection of the nucleic acid in the cell, for example, but not limited to particle gun technology, colloidal dispersion systems, electroporation, vectors, and the like. In some embodiments, the use of inducible constructs [e.g., Tet on/off system (Clontech, Mountain View, Calif., USA)] would allow control of the amount of BMP-7 produced by cells. In some embodiments, lentivirus (Clontech) and/or baculovirus systems and/or other viral vector systems could be used for delivery of a BMP-7 gene construct.

In its broadest sense, a “delivery system,” as used herein, is any vehicle capable of facilitating delivery of a nucleic acid (or nucleic acid complex) to a cell and/or uptake of the nucleic acid by the cell. Other example delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell).

The term “transfection,” as used herein, refers to the introduction of a nucleic acid into a cell. “Transfection” as used herein is intended to cover introduction of a nucleic acid into a eukaryotic cell. “Transfection” as used herein is also intended to encompass “transformation” (introduction of a nucleic acid into a prokaryotic cell) and “transduction” (introduction of a nucleic acid into a cell using a viral vector). In some embodiments, transfection may be used to genetically modify a cell. For example, a cell may be transfected with a nucleic acid coding for BMP-7 or functional variants or functional fragments thereof and/or a BMP-7 agonist. In some embodiments, the genetically modified cell may overexpress the BMP-7 or functional variants or functional fragments thereof and/or a BMP-7 agonist. The terms “transformation” and “transduction” are also used herein according to their ordinary meaning. Transfection may be accomplished by a variety of means known to the art. Such methods include, but are not limited to, particle bombardment mediated transformation (e.g., Finer et al., Curr. Top. Microbiol. Immunol., 240:59 (1999)), viral infection (e.g., Porta and Lomonossoff, Mol. Biotechnol. 5:209 (1996)), microinjection, electroporation, and liposome-mediated delivery. Standard molecular biology techniques are common in the art (See e.g., Sambrook, J. et al., Molecular Cloning: A Laboratory Manual, 2^{nd} ed., Cold Spring Harbor Laboratory Press, New York (1989)).

For instance, in one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry DNA or other nucleic acids into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of the nucleic acid (or nucleic acid complex) into the cell. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which range in size from 0.2 to 4.0 microns can encapsulate large macromolecules within the aqueous interior and these macromolecules can be delivered to cells in a biologically active form (Fraley, et al., Trends Biochem. Sci., 6:77 (1981)).

Lipid formulations for transfection and/or intracellular delivery of nucleic acids are commercially available, for instance, from QIAGEN, for example as EFFECTENE® (a non-liposomal lipid with a special DNA condensing enhancer) and SUPER-FECT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985).

Electroporation may be used, in another set of embodiments, to deliver a nucleic acid (or nucleic acid complex) to the cell. Electroporation, as used herein, is the application of electricity to a cell in such a way as to cause delivery of the nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by one of ordinary skill in the art.

In yet another set of embodiments, the nucleic acid may be delivered to the cell in a vector. In its broadest sense, a “vector” is any vehicle capable of facilitating the transfer of the nucleic acid to the cell such that the nucleic acid can be processed and/or expressed in the cell. Preferably, the vector transports the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors. Examples of such cells have been previously described.

In general, vectors useful in the invention include, but are not limited to, plasmids, phagemids, viruses, other vehicles derived from viral or bacterial sources that have been manipulated by the insertion or incorporation of the nucleotide sequence (or precursor nucleic acid) of the invention. Viral vectors useful in certain embodiments include, but are not limited to, nucleic acid sequences from the following viruses: lentiviruses, retroviruses such as Moloney murine leukemia viruses, Harvey murine sarcoma viruses, murine mammary tumor viruses, and Rous sarcoma viruses; adenovirus, or other adeno-associated viruses; SV40-type viruses; polyoma viruses; Epstein-Barr viruses; papilloma viruses; herpes virus; vaccinia virus; polio viruses; and RNA viruses such as retroviruses. One can readily employ other vectors not named but known to the art.

Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) can be found in Kriegler, M., Gene Transfer and Expression, A Laboratory Manual, W.H. Freeman Co., New York (1990) and Murry, E. J. Ed., Methods in Molecular Biology, Vol. 7, Humana Press, Inc., Cliffton, N.J. (1991), both hereby incorporated by reference.

Another example of a virus for certain applications is the adeno-associated virus, which is a double-stranded DNA virus. The adeno-associated virus can be engineered to be replication-deficient and is capable of infecting a wide range of cell types and species. The adeno-associated virus further has advantages, such as heat and lipid solvent stability; high transduction frequencies in cells of diverse lineages, including hemopoietic cells; and/or lack of superinfection inhibition, which may allow multiple series of transduction. AAV-vectors have been used for delivery of BMP-7 to mammalian cell types, for example, as described in Zhonghua Yi Xue Za Zhi (2006) 86(8):544-8; Zhejiang Da Xue Xue Bao Yi Xue Ban (2010) 39(1):71-8; Mol. Biotechnol. (2010) 46(2):118-26; Acta Pharniacol. Sin. (2007) 28(6):839-49; Acta Pharmacol. Sin. (2007) 28(6):839-49; J. Endod. (2007) 33(8):930-5; Spine (Phila Pa. 1976) (2003) 28(18):2049-57; Expert Rev. Mol. Med. (2010) 12:e18; J. Orthop. Res. (2010) 28(3):412-8; and Int. J. Artif. Organs. (2010) 33(6):339-47; each of which is incorporated herein by reference.

Another vector suitable for use with the invention is a plasmid vector. Plasmid vectors have been extensively described in the art and are well-known to those of skill in the art. See e.g., Sambrook, et al., Molecular Cloning: A Laboratory Manual, Second Edition, Cold Spring Harbor Laboratory Press, 1989. These plasmids may have a promoter compatible with the host cell, and the plasmids can express a polypeptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence. These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell. The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, T. et al., Science 236:1237 (1987), incorporated herein by reference. Promoter and enhancer elements have been isolated from a variety of eukaryotic sources including genes in plant, yeast, insect and mammalian cells and viruses (analogous control elements, i.e., promoters, are also found in prokaryotes).

The selection of a particular promoter and enhancer depends on what cell type is to be used and the mode of delivery. Our results have shown that the CMV promoter works well in HPTCs. For example, a wide variety of promoters have been isolated from plants and animals, which are functional not only in the cellular source of the promoter, but also in numerous other plant and/or animal species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus (CaMV). Promoters that have been isolated and reported for use in plants include ribulose-1,3-biphosphate carboxylase small subunit promoter, phaseolin promoter, etc.

Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Thus, a variety of promoters and regulatory elements may be used in the expression vectors of the present invention. For example, in some preferred embodiments an inducible promoter is used to allow control of nucleic acid expression through the presentation of external stimuli (e.g., environmentally inducible promoters). Thus, the timing and amount of nucleic acid expression may be controlled. Non-limiting examples of expression systems, promoters, inducible promoters, environmentally inducible promoters, and enhancers are described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472.

As used herein, an “expression element” can be any regulatory nucleotide sequence, such as a promoter sequence or promoter-enhancer combination, which facilitates the efficient expression of the nucleic acid. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (HPRT), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences. Because a patient may be exposed to the agents used for induction, use of a metallothionein promoter might not desirable. Preferred is an agent that is only used when the promoter should be switched off and is relatively non-toxic. An example is the Tet-off system from Clontech (Mountain View, Calif.), where tetracycline is used to switch off gene expression.

In another set of embodiments, homologous recombination can be used to alter the expression of BMP-7. In some instances, recombination can be used to alter a promoter of BMP-7 expression. In other instances, the BMP-7 gene itself can be altered. In some embodiments, the promoter for a BMP-7 protein can be used to monitor the expression of the BMP-7 protein, for example by using the promoter for a BMP-7 protein to drive the expression of an indicator such as a fluorescent protein.

Using any gene transfer technique, such as the above-listed techniques, an expression vector harboring the nucleic acid may be transfected into a cell to achieve temporary or prolonged expression. Any suitable expression system may be used, so long as it is capable of undergoing transfection and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (GFP) is used to allow simple selection of transfected cells and to monitor expression levels. Non-limiting examples of such vectors include Clontech's “Living Colors Vectors” pEYFP and pEYFP-C1.

In some cases, a selectable marker may be included with the nucleic acid being delivered. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

In some embodiments, the BMP-7 may be overexpressed in a cell. The term “overexpressed” or “overexpression” means that the BMP-7 or functional variants or functional fragments thereof and/or a BMP-7 enhancer is expressed at a level greater than the expression level observed in a wild type cell. For example, a renal proximal tubule cell containing an exogenous BMP-7 open reading frame may overexpress BMP-7 relative to a reference renal proximal tubule cells that contains only the native chromosomal BMP-7 open reading frame. In some embodiments, a cell may be genetically modified to overexpress BMP-7 or functional variants or functional fragments thereof and/or a BMP-7 agonist.

In some embodiments, BMP-7 or functional variants or functional fragments thereof and/or a BMP-7 agonist can be delivered to cells using a controlled release strategy. In some embodiments, the BMP-7 may be released from a membrane (e.g., the reabsorption membrane). In other embodiments, the BMP-7 may be released from elsewhere in the device. For example, in some cases, the BMP-7 may be released from a tube of the device, a housing, a channel, etc. For example, in some embodiments, the BMP-7 or functional variants or functional fragments thereof and/or a BMP-7 agonist may be embedded in or absorbed in a material (e.g., a polymeric material) and/or coated onto a material in the device. In another embodiment, BMP-7 or functional variants or functional fragments thereof and/or a BMP-7 agonist may be incorporated into a matrix, such as a hydrogel. For example, in some embodiments, BMP-7 or functional variants or functional fragments thereof and/or a BMP-7 agonist may be encapsulated in particles (e.g., microparticles or nanoparticles): In one embodiment, BMP-7 or functional variants or functional fragments thereof and/or a BMP-7 agonist may be encapsulated in polymer-inorganic microparticles [Pitukmanorom et al. Advanced Materials (2008) 20, 3504-3509, incorporated herein by reference].

In some embodiments, particles loaded with BMP-7 or functional variants or functional fragments thereof and/or a BMP-7 agonist may be incorporated into the semi-permeable membrane to provide for controlled release of the BMP-7 or functional variants or functional fragments thereof and/or a BMP-7 agonist. In some cases, the membrane may have a layered configuration where the cells are attached to the exposed surface of a first layer and a second layer encapsulating the BMP-7 or functional variants or functional fragments thereof and/or a BMP-7 agonist containing microspheres is disposed between the first layer and a third layer. Such a configuration can allow substances such as nutrients and ions to penetrate through the membrane while, for example, BMP-7-loaded particles can provide for the release of BMP-7 into the filtrate to provide an environment to keep the HTPCs viable and polarized.

The particles may be any suitable size. For example, in some cases the particles may have an average particle size greater than 50 nm, greater than 200 nm, greater than 500 nm, greater than 1 micron, greater than 10 microns, or greater than 100 microns. In some cases, the particles have an average particle size between 50 nm and 100 microns or in other cases between about 100 nm and 10 microns. The particle size may be chosen to elicit certain properties (i.e., release rate of an agent, degradation rate, agent loading capacity, etc.). As used herein, “particle size” refers to the largest characteristic dimension (i.e. of a line passing through the geometric center of the particle e.g., diameter) that can be measured along any orientation of a particle (e.g., a polymer particle). The particle-size distribution may be reported as the weight percentage of particles retained on each of a series of standard sieves of decreasing size, and the percentage of particles passed of the finest size. That is, the average particle size may correspond to the 50% point in the weight distribution of particles.

The particles may be formed from any suitable material. For example, in some embodiments, the particles may be formed from polymers and/or inorganic materials. The materials include, but are not limited to, the numerous materials that have been used for controlled drug release and are known to those of ordinary skill in the art. In some cases, the particles may be non-degradable. In some embodiments, the particles may be degradable. For example, the particles may be formed from degradable polymers such as polylactic acid, polyglycolic acid, polycaprolactone, and copolymers and blends thereof. Other degradable polymer are known to those of ordinary skill in the art.

Particles loaded with BMP-7 or functional variants or functional fragments thereof and/or a BMP-7 agonist may be fabricated by any of a number of known techniques. For example, particles loaded with BMP-7 or functional variants or functional fragments thereof and/or a BMP-7 agonist may be fabricated by emulsion techniques (e.g., double emulsion) or spray drying.

In some embodiments, a matrix such as a membrane material or other component of a fluidic device may be loaded directly with BMP-7 or functional variants or functional fragments thereof and/or a BMP-7 agonist by adsorption to the membrane material, without the involvement of any particles. Alternatively, BMP-7 can be released from all other parts of the device, e.g. housing or tubing. In some embodiments, loading may be achieved by pre-adsorption of the BMP-7 or functional variants or functional fragments thereof and/or a BMP-7 agonist to the housing/tubing materials or by incorporating BMP-7-loaded particles (e.g., nano/microparticles), as described above. In some embodiments, release of BMP-7 or functional variants or functional fragments thereof and/or a BMP-7 agonist may not require cells and thus could be achieved, for example, using a standard artificial kidney (e.g. hemodialysis machine).

In some embodiments, a membrane on which the renal proximal tubule cells grow may release BMP-7 or functional variants or functional fragments thereof and/or a BMP-7 agonist at a controlled rate sufficient to produce a desired concentration of BMP-7 or functional variants or functional fragments thereof and/or a BMP-7 agonist. For example, in blood filtration devices where fluid enters and exits the device, the rate of BMP-7 release may be configured to provide a concentration of BMP-7 in the effluent of at least 0.001 nM, at least 0.01 nM, at least 0.05 nM, at least 0.1 nM, at least 0.5 nM, at least 1 nM, at least 2 nM, at least 5 nM, at least 10 nM, at least 20 nM, or at least 50 nM. In some embodiments, the concentration of BMP-7 in the effluent may have a concentration between 0.01 nM and 5 nM, between, 0.01 nM and 2 nM, between, 0.05 nM and 2 nM, between 0.1 nM and 2 nM, or between 0.5 nM and 2 nM.

In some embodiments, the function of BMP-7 can be increased by appropriate use of an agonist. In some embodiments, an agonist may be delivered from the device without BMP-7 in order to enhance the function of residual endogenous BMP-7 in the patient. For example, kielin/chordin-like protein (KCP) or functional variants or functional fragments thereof may be used as a BMP-7 agonist. As used herein, “agonist” generally refers to a molecular species that binds to a receptor of a cell and stimulates a response by the cell. “Agonist” may also refer to a molecular species that enhances the effect of a signaling molecule (i.e., BMP-7). In some embodiments, the agonist may bind to the signaling molecule. In other embodiments, the agonist may bind to the signaling molecule receptor. In some embodiments, one or more agonists may be delivered using the techniques described above.

It should be understood that KCP refers to a human protein encoded by the amino acid sequence of SEQ ID NO. 3 or 5. In some embodiments, the amino acid sequence of KCP, is SEQ ID NO. 3. In some embodiments, the amino acid sequence of KCP is SEQ ID NO. 5. In certain embodiments, rather than using KCP, a functional variant or functional fragment thereof may be employed. In some embodiments, the amino acid sequence of KCP may be coded for by the nucleic acid sequence of SEQ ID NO. 4 or 6. In certain embodiments, the amino acid sequence of KCP may be coded for by the complement of a nucleic acid sequence that hybridizes to the nucleic acid sequence of SEQ ID NO. 4 or 6 under high stringency conditions. Such nucleic acids may be DNA, RNA, composed of mixed deoxyribonucleotides and ribonucleotides, or may also incorporate synthetic non-natural nucleotides. Various methods for determining the expression of a nucleic acid and/or a polypeptide in normal and tumor cells are known to those of skill in the art.

Functional variants may result from, for example, genetic polymorphism or from human manipulation. Biologically active variants and fragments (i.e. functional variants and functional fragments) of a KCP protein of the invention will have at least about 75%, 80%, 85%, 90%, 91%, 92%, 93%, 94%, 95%, 96%, 97%, 98%, 99% or more sequence identity to one of the amino acid sequences for the human KCP protein as determined by sequence alignment programs and parameters described elsewhere herein. A biologically active variant of a protein consistent with an embodiment of the invention may differ from that protein by as few as 1-15 amino acid residues, as few as 1-10, such as 6-10, as few as 5, as few as 4, 3, 2, or even 1 amino acid residue.

In some embodiments, a functional variant or fragment of SEQ ID NO. 3 or 5 typically will share with SEQ ID NO. 3 or 5, respectively, at least 75% amino acid identity, in some instances at least 80% amino acid identity, in some instances at least 90% amino acid identity, in some instances at least 95% amino acid identity, in some instances at least 96% amino acid identity, in some instances at least 97% amino acid identity, in some instances at least 98% amino acid identity, and in some instances at least 99% amino acid identity.

In some embodiments, a BMP-7-producing device can deliver BMP-7 not only to the cells within the device but also to a patient whose circulation system is fluidly connected to the device. Without wishing to be bound by any theory, it is believed that BMP-7 has anti-inflammatory, cytoprotective, and anti-fibrotic effects on kidney cells. Thus, administration of BMP-7 to patient may be used to treat ailments of the kidney. For example, in some cases, BMP-7 may be used to prevent the progression to chronic renal disease. In some embodiments, methods of the invention can be used treatment of patients with acute renal failure (ARF). It has been shown in animal experiments that BMP-7 improves kidney recovery. ARF patients are hospitalized and usually treated for prolonged time periods or continuously with artificial kidneys, which facilitates delivery of relatively low concentrations of BMP-7 over prolonged time periods. However, the overall duration of the treatment is limited to a period of about 1-2 weeks and this also limits the overall costs of the treatment, which may pose certain challenges in case of chronic kidney disease.

Advantageously, a BAK or dialysis device capable of delivering BMP-7 may be used to deliver BMP-7 continuously, thus circumventing a conventional treatment strategy involving multiple administrations of BMP-7. In some embodiments, BMP-7 or functional variants or functional fragments thereof and/or a BMP-7 agonist may administered to a patient in need thereof. For example, BMP-7 or functional variants or functional fragments thereof and/or a BMP-7 agonist may be used to improve kidney recovery after acute injury (e.g., in acute renal failure), inhibit progression of chronic kidney disease (CKD), and/or provide beneficial effects for non-renal conditions often associated with CKD (e.g., renal osteodystrophy, for example, in bone disease and/or vascular calcification).

In some embodiments, BMP-7 or functional variants or functional fragments thereof and/or a BMP-7 agonist may administered to a patient in a therapeutic amount corresponding to or exceeding physiological levels of BMP-7. For example, in some embodiments, BMP-7 or functional variants or functional fragments thereof and/or a BMP-7 agonist may be administered to a patient at a concentration of between 100 ng/kg/day to 500 ng/kg/day, in some embodiments between 100 ng/kg/day to 400 ng/kg/day, or in some embodiments between 100 ng/kg/day to 300 ng/kg/day. In some cases, BMP-7 or functional variants or functional fragments thereof and/or a BMP-7 agonist may be administered to a patient at a concentration of at least 100 ng/kg/day, in some embodiments at least 200 ng/kg/day, in some embodiments at least 300 ng/kg/day, in some embodiments at least 400 ng/kg/day, or in some embodiments at least 500 ng/kg/day. In some embodiments, BMP-7 or functional variants or functional fragments thereof and/or a BMP-7 agonist may administered to a patient in a therapeutic amount that aims to improve the performance and functionality of renal cells. For example, in some embodiments, BMP-7 or functional variants or functional fragments thereof and/or a BMP-7 agonist may be administered to a patient at a concentration of between 10 mg/kg/day to 50 mg/kg/day, in some embodiments between 10 mg/kg/day to 40 mg/kg/day, or in some embodiments between 10 mg/kg/day to 30 mg/kg/day. In some cases, BMP-7 or functional variants or functional fragments thereof and/or a BMP-7 agonist may be administered to a patient at a concentration of at least 10 mg/kg/day, in some embodiments at least 20 mg/kg/day, in some embodiments at least 30 mg/kg/day, in some embodiments at least 40 ms/kg/day, or in some embodiments at least 50 μg/kg/day.

The following examples are intended to illustrate certain embodiments of the present invention, but do not exemplify the full scope of the invention.

### Example 1

This example demonstrates that human recombinant BMP-7 inhibits tubule formation and improves performance of renal cells applied in bioartificial kidneys.

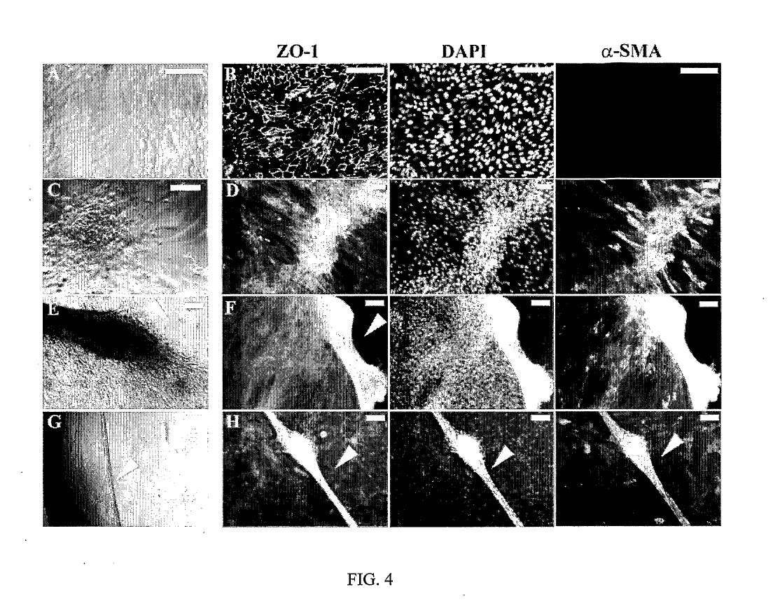

Firstly, it was investigated whether treatment with BMP-2 or BMP-7 inhibited the disruption of epithelia formed by HPTCs. Cell behavior was monitored during an extended time period of 4 weeks for each experiment. In untreated controls (FIG. 4), increasing numbers of α-SMA-expressing myofibroblasts during the monitoring period and cell aggregate formation were observed, as well as de-differentiation, rearrangement and disruption of the epithelium. FIG. 4 shows formation and disruption of epithelia formed by HPTCs. The left-hand panels (A, C, E, G) show differential interference contrast (DIC) or phase contrast images of live HPTCs. Rows B, D, F and H (the three panels in each row display the same field of cells) show ZO-1 and α-SMA immunofluorescence patterns and the corresponding DAPI staining as indicated. (A, B) Properly differentiated epithelia were formed within the first week after cell seeding. (C-F) During the next 1-2 weeks, increasing numbers of α-SMA-expressing myofibroblasts appeared. Large cell aggregates were formed, and the epithelium in the surroundings of such cell aggregates de-differentiated (note the absence of chicken wire-like ZO-1 patterns in D and F) and became rearranged and disrupted (some areas devoid of cells are labeled with arrowheads in E and F). (G, H) Rearrangements led to the formation of renal tubules (marked by arrowheads) on the substrate surface. Scale bars: 100 μm (A-E), 200 μm (F-H). These processes led to the formation of renal tubules on the substrate surface, and the observations were in agreement with previous results [Zhang et al. The impact of extracellular matrix coatings on the performance of human renal cells applied in bioartificial kidneys. Biomaterials (2009) 30, 2899; Zhang et al. Generation of easily accessible human kidney tubules on two-dimensional surfaces in vitro. J. Cell Mol. Med. (2010), epub ahead of print].

### Effects of BMP-7 on the Maintenance of Epithelia Formed by HPTCs

The following concentrations of BMP-7 were tested: 4 nM, 3 nM, 2 nM, 1 nM and 0.5 nM (Table 1). In most of the experiments, BMP-7 was added during cell seeding, and from then on, the cells were constantly kept in BMP-7-supplemented medium. In the case of treatment with 4 nM of BMP-7, the growth factor was added either already during cell seeding or only later after the epithelium formation, since the possibility could not be excluded that BMP-7 compromised the initial formation of the epithelium.

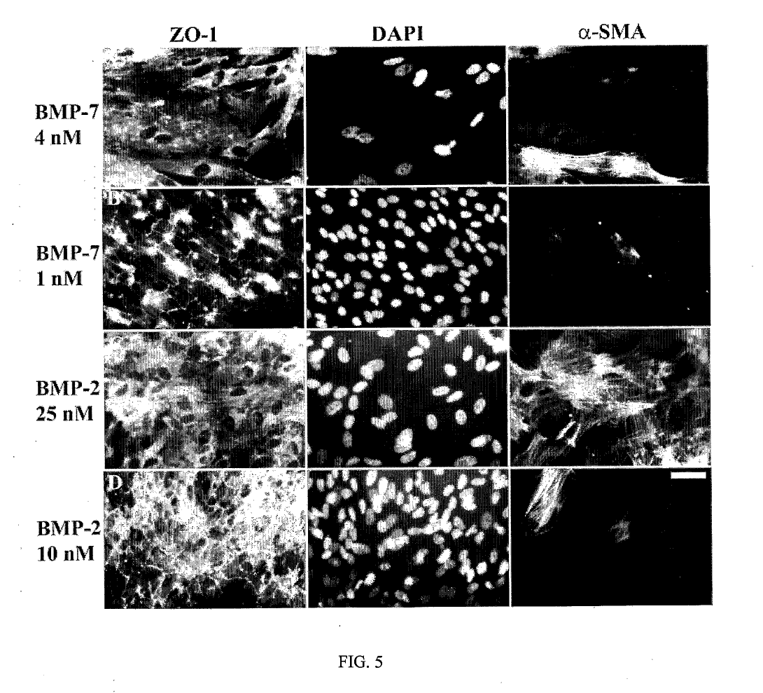

Monolayer formation and maintenance during the monitoring period of 4 weeks were assessed, along with the degree of epithelial differentiation via the ZO-1 immunostaining patterns (Table 1). Classification of ZO-1 immunostaining patterns was performed as described [Zhang et al. The impact of extracellular matrix coatings on the performance of human renal cells applied in bioartificial kidneys. Biomaterials (2009) 30, 2899]. Typical chicken wire-like ZO-1 immunostaining patterns indicating extensive tight junction formation and the formation of a properly differentiated epithelium were classified as types 4 or 5. More diffuse ZO-1 immunostaining patterns were classified as types 1-3, and these patterns indicated insufficient epithelial differentiation and tight junction formation. Type 1-2 patterns revealing insufficient epithelial differentiation were obtained with most of the BMP-7 concentrations tested (Table 1). High numbers of α-SMA-expressing cells were present in samples displaying insufficient epithelial differentiation (FIG. 5 A). FIG. 5 shows effects of different concentrations of BMP-7 and BMP-2. Representative images of HPTCs exposed to different concentrations of BMP-7 and BMP-2 are shown. Imaging was performed 2 weeks after cell seeding. The three panels in each row (A-D) display the same field of cells. The panels show ZO-1 and α-SMA immunofluorescence patterns and the corresponding DAPI staining, as indicated. (A, C) The monolayers display a relatively low cell density, high numbers of myofibroblasts and insufficient tight junction formation at high concentrations of BMP-7 and BMP-2. (B, D) Epithelial differentiation was improved at lower concentrations of BMP-7 (1 nM) and BMP-2 (10 nM), and lower numbers of α-SMA-positive cells were observed. Scale bar: 50 μm. However, formation of cell aggregates and tubules did not occur under these conditions, and the monolayer was maintained in most samples until the end of the monitoring period (Table 1). An exception in this regard were the samples treated with 0.5 nM of BMP-7, where disruption of the monolayer occurred after 1 week. Early disruption took place also when 4 nM of BMP-7 were applied after monolayer formation (Table 1).

In contrast, a well-differentiated epithelium was obtained with 1 nM of BMP-7 (Table 1, FIG. 5B), and low numbers of α-SMA-expressing cells were observed under these conditions. No tubule formation or monolayer disruption was observed over the period of 4 weeks, and intact epithelia could be maintained during the entire monitoring period (Table 1). These results showed that treatment with 1 nM of BMP-7 did not promote the accumulation of myofibroblasts, inhibited the disruption of epithelia, and substantially improved cell performance. The study also revealed that the effects of BMP-7 were strongly concentration-dependent.

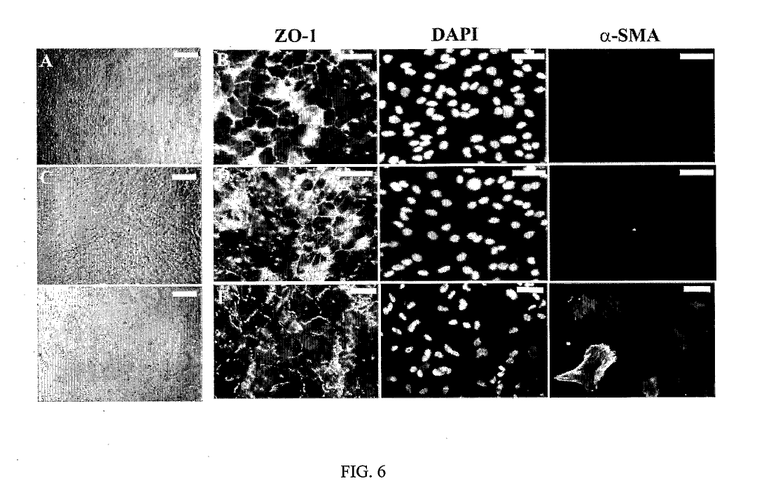

To address the issue of variability between different batches of the primary cells derived from different patients, the experiments with 1 nM of BMP-7 were repeated using different batches of HPTCs. The results were consistent for the different batches of cells; well-differentiated epithelia could be maintained during the entire monitoring period of 4 weeks in all cases (FIG. 6). FIG. 6 shows treatment with 1 nM of BMP-7 improved the long-term maintenance of epithelia. The left-hand panels (A, C, E) show DIC and phase contrast images of live HPTCs. Rows B, D and F (the three panels in each row display the same field of cells) show ZO-1 and α-SMA immunofluorescence patterns and the corresponding DAPI staining, as indicated. Rows A and B, C and D and E and F display cells from three different batches of HPTCs. All images were captured after 4 weeks of in vitro culture. In all cases, properly differentiated epithelia could be maintained for this time period, and overall only a few α-SMA positive cells were observed. Higher numbers of α-SMA-positive cells, lower cell density and zigzag ZO-1 staining patterns indicated a slightly compromised epithelial differentiation in the cell batch displayed in rows E and F. Scale bars: 200 μm (A, C, E) and 50 μm (B, D, F).

### Effects of BMP-2-Treatment

In a next series of experiments, the effects of BMP-2 were tested. Inhibition of tubulogenesis in in vitro systems had been observed previously after applying various concentrations of BMP-2, ranging from 1 nM to 25 nM [Grisaru et al. Glypican-3 modulates BMP- and FGF-mediated effects during renal branching morphogenesis. Dev Biol. (2001) 231, 31; Piscione et al. BMP-2 and OP-1 exert direct and opposite effects on renal branching morphogenesis. Am. J. Physiol. (1997) 273, F961; Piscione et al. BMP7 controls collecting tubule cell proliferation and apoptosis via Smad1-dependent and -independent pathways. Am. J. Physiol. Renal Physiol. (2001) 280, F19]. As it was unclear which range of concentrations might be suitable for this experimental system, different concentrations were tested within this range.

As summarized in Table 1, at high concentrations of BMP-2 (25 nM, 20 nM, 15 nM and 12 nM), disruption of the monolayer was inhibited when cells were consistently exposed to BMP-2. However, proper epithelial differentiation did not occur, and high numbers of α-SMA-positive cells were observed (FIG. 5 C). Disruption of the monolayer was not inhibited when BMP-2 was added only after the epithelium formation (20 nM). Application of lower concentrations of BMP-2 (8 nM, 5 nM and 1 nM) resulted in slightly improved epithelial differentiation, but disruption of the epithelium was not inhibited (Table 1). Results obtained with 1 nM of BMP-2 showed some variability between the five replicas analyzed during the first experimental series. This experimental series was repeated with another batch of cells, and a relatively high degree of variability was observed again.

The best results in terms of epithelial differentiation were obtained with 10 nM of BMP-2 (Table 1, FIG. 5D). Disruption of the monolayer was delayed at this concentration of BMP-2 as compared to the controls. However, variable results were also obtained here; inhibition of epithelial disruption was observed in some of the replicas during one experimental series, while cell aggregate and tubule formation occurred in others. The experiments with 10 nM of BMP-2 were repeated with three different batches of HPTCs, but a relatively high degree of variability was observed in all cases, and cell aggregate and tubule formation always occurred in some of the replicas.

### Quantification of α-SMA Expression

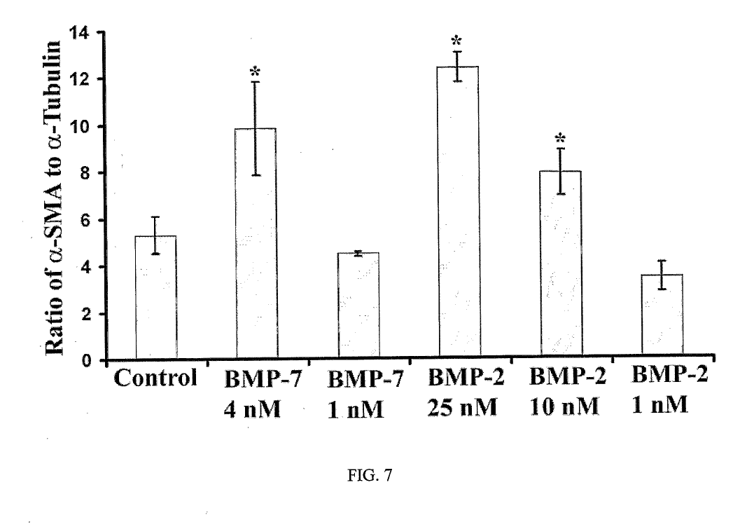

A consistent observation throughout the different series of experiments was the presence of increasing numbers of α-SMA-positive cells at higher concentrations of BMP-2 and BMP-7 (FIG. 5). Immunoblotting was performed in order to quantify the levels of α-SMA expression at different concentrations of BMP-2 or BMP-7 after two weeks of treatment. The levels of α-SMA expression were not significantly changed in cultures treated with 1 nM of BMP-2 or BMP-7, respectively, as compared to untreated controls (FIG. 7). FIG. 7 shows quantification of α-SMA expression at different concentrations of BMP-7 and BMP-2. HPTCs were exposed to the different concentrations of BMP-7 or BMP-2 indicated (in the x-axis) or left untreated (control). In each case, proteins were extracted from 3 replicate of cultures after 2 weeks of in vitro culture, and each extract was loaded onto a separate lane of a gel. Immunoblotting was used to detect α-SMA- and α-tubulin-specific bands. Band intensities were determined, and the ratios of α-SMA to α-tubulin band intensities are indicated by the bars (average+/−standard deviation). The relative levels of α-SMA expression in cultures treated with 1 nM of BMP-7 or BMP-2 were not significantly different from those of the control (p>0.05). In contrast, significantly increased α-SMA expression levels were observed when 4 nM of BMP-7 or 25 nM and 10 nM of BMP-2 were applied (as compared to the control, and the cultures treated with 1 nM of BMP-7 or BMP-2 (p<0.05)). Significantly higher levels of α-SMA were observed after treatment with 4 nM of BMP-7 or 10 nM and 25 nM of BMP-2. The results showed that the occurrence of α-SMA-expressing myofibroblasts was not inhibited by the treatment with low concentrations of BMP-2 and BMP-7, but that higher concentrations of these growth factors increased the levels of α-SMA expression.

TABLE 1
HPTC performance at different concentrations
of BMP-2 and BMP-7.

| Growth | Concentration | Monolayer | ZO-1 immunostaining |
|---|---|---|---|
| Factor | (nM) | formation | pattern |
| BMP-2 | 25 | +, until week 4 | 1-2, until week 3 |
| | 20 | +, until week 4 | 1-2, until week 4 |
| | 20** | +, until week 1 | 1-2, until week 1 |
| | 15 | +, until week 4 | 1-2, until week 4 |
| | 12 | +, until week 3 | 1-2, until week 3 |
| | 10 | +, until week 3* | 4, until week 1 * |
| | 8 | +, until week 2 | 2-3, until week 2 |
| | 5 | +, until week 1 | 3, until week 1 |
| | 1 | +, until week 1* | 3, until week 1* |
| BMP-7 | 4 | +, until week 4 | 1-2, until week 4 |
| | 4** | +, until week 1 | 1-2, until week 1 |
| | 3 | +, until week 4 | 1-2, until week 4 |
| | 2 | +, until week 4 | 1-2, until week 4 |
| | 1 | +, until week 4 | 4-5, until week 4 |
| | 0.5 | +, until week 1 | 1-2, until week 1 |

“+” indicates formation of a confluent monolayer. “Until week x” refers to the week until which the monolayer remained intact, or the indicated ZO-1 staining pattern was observed (i.e. disrupted thereafter). The ZO-1 staining patterns was classified as described previously [Zhang et al. The impact of extracellular matrix coatings on the performance of human renal cells applied in bioartificial kidneys. Biomaterials (2009) 30, 2899]. Only type 4 and type 5 staining patterns indicate proper epithelial differentiation and tight junction formation. At least three replicates were monitored in each case, and most of the experimental series were repeated at least twice with different batches of cells.
*Results variable between different wells and cell batches.
**Growth factor added after monolayer formation.

### Example 2

This example provides the materials and methods for the experiments described in Examples 1 and 2.

### Cell Culture

HPTCs were obtained from ScienCell Research Laboratories (Carlsbad, Calif., USA). Different batches of HPTCs were obtained and cultivated in basal epithelial cell medium supplemented with 2% fetal bovine serum (FBS) and 1% epithelial cell growth supplement (all components obtained from ScienCell Research Laboratories). All cell culture media used were supplemented with 1% penicillin/streptomycin solution (ScienCell Research Laboratories), and all cells were cultivated at 37° C. in a 5% CO₂ atmosphere. The seeding density was 5×10⁴ cells/cm². Experiments with were performed with 24-well cell culture plates (Nunc, Naperville, Ill., USA). All substrates used for the cultivation of HPTCs were coated with human laminin (100 μg/ml, Sigma, St. Louis, Mo., USA) (20). For all the experiments, the cell culture medium was exchanged every 2 days during the experimental series. Staining of living cells with 4′,6′-diamidino-2′-phenylindole (DAPI, Merck, Darmstadt, Germany) and formaldehyde fixation were performed [Zhang et al. The impact of extracellular matrix coatings on the performance of human renal cells applied in bioartificial kidneys. Biomaterials (2009) 30, 2899].

Treatment with BMP-2 and BMP-7

BMP-7 and BMP-2 (Miltenyi Biotec, Bergisch-Gladbach, Germany) were obtained in the lyophilized form, and solubilized in phosphate buffered saline (PBS). They were added at the relevant concentrations to the cell culture media. Growth factor concentrations are indicated in ng/ml as well as in nM to facilitate comparisons with previous studies. BMP-7 has variable molecular weights due to glycosylations [Sampath et al. Bovine osteogenic protein is composed of dimers of OP-1 and BMP-2A, two members of the transforming growth factor-beta superfamily. J. Biol. Chem. (1990) 265, 13198], and for our calculations, we assumed an average molecular weight of 25 kDa. 4 nM (100 ng/ml), 3 nM (75 ng/ml), 2 nM (50 ng/ml), 1 nM (25 ng/ml) and 0.5 nM (12.5 ng/ml) of BMP-7 were tested. For BMP-2, concentrations of 25 nM (650 ng/ml), 20 nM (520 ng/ml), 15 nM (390 ng/ml), 12 nM (312 ng/ml), 10 nM (260 ng/ml), 8 nM (208 ng/ml), 5 nM (130 ng/ml) and 1 nM (26 ng/ml) were analyzed. In the corresponding experimental series, growth factors were added during cell seeding, and cells were constantly kept in growth factor supplemented medium. In a separate series of experiments, BMP-7 (4 nM) and BMP-2 (20 nM) were added only after monolayer formation.

### Immunostaining and Imaging

Immunostaining was performed as described [Zhang et al. The impact of extracellular matrix coatings on the performance of human renal cells applied in bioartificial kidneys. Biomaterials (2009) 30, 2899]. Rabbit anti-ZO-1 (Invitrogen, Carlsbad, Calif., USA) and mouse anti-α-SMA (Abeam, Cambridge, UK) antibodies were used for HPTCs. The primary antibodies were detected using Alexa Fluor 488-conjugated anti-rabbit (Invitrogen) and TRITC-conjugated anti-mouse (Invitrogen) secondary antibodies. Following immunostaining, cell nuclei were stained with DAPI and the cells were mounted with vectashield (Vector Laboratories, Burlingame, Calif.) for microscopy. The Zeiss AxioObserver Z1 microscope (Carl Zeiss, Jena, Germany) with the Zeiss AxioVision imaging software was used for imaging. Adobe Photoshop CS3 and ImageJ were used to arrange the images. The different types of ZO-1 immunostaining patterns were classified as described [Zhang et al. The impact of extracellular matrix coatings on the performance of human renal cells applied in bioartificial kidneys. Biomaterials (2009) 30, 2899].

### Immunoblotting

Cells were lysed in 100-μl lysis buffer containing 20 mM of Tris-Cl, 2 mM of ethylenediaminetetraacetic acid (EDTA), 150 mM of sodium chloride, 10% of glycerol, 1% of Triton X-100, and 1 mM of a mixture of protease inhibitors (PMSF). Lysates were vortexed and centrifuged for 10 min at 12,000×g. The protein concentration of the supernatants was measured using the bicinchoninic acid (BCA) Protein Assay Kit (Pierce, Rockford, Ill., USA). Equal amounts of protein were loaded onto a NuPage precasted gel (4-12%, Invitrogen), and the Spectra Multicolor Broad Range Protein Ladder (Fermentas, Hanover, Md., USA) was used as the molecular weight marker. After electrophoresis, proteins were transferred to iBlot membranes (Invitrogen), which were then blocked in tris(hydroxymethyl)aminomethane (Tris) buffered saline (TBS) containing 0.05% of Tween-20 (TBS-T) and 3% of bovine serum albumin (Sigma). Blocking was performed at room temperature for 1 h. The membranes were then incubated overnight at 4° C. with mouse anti-α-SMA and rabbit anti-α-tubulin antibodies (Abeam) (dilution=1:5000). Following washing with TBS-T, sheep anti-mouse and donkey anti-rabbit antibodies (peroxidase conjugated, 1:10000) were added to the membranes. Both primary and secondary antibodies were diluted in blocking buffer. The membranes were washed with TBS-T, and the blots were developed using the ECL detection kit (GE Healthcare, Chalfont St. Giles, Buckinghamshire, UK). The chemiluminescence signal was captured on X-ray films, which were scanned and analyzed using Adobe Photoshop CS3. The paired t-test was used for statistics.

### Example 3

This example describes baculoviral cloning of BMP-7.

BMP-7 cDNA along with CMV promoter was amplified from A0309 Human BMP-7 Full Length ORF Mammalian Free Expression from GeneCopoeia, Inc. (Rockville, Md., USA) (Cat # EX-A0309-M02) using polymerase chain reaction (PCR). SEQ ID NO. 2 is the nucleic acid sequence of BMP-7 in this vector. The primers used for the PCR amplification contained overhangs with restriction enzymes (NotI and KpnI). The PCR product and the baculoviral vector (pFastBac1, Invitrogen Corporation) were digested using NotI and KpnI and conventional ligation was carried out to obtain P_{CMV} BMP-7 in pFastBac1 Vector. The ligation product was transformed into DH 5α competent cells (Invitrogen). Clones were verified using restriction digestion. Selected positive clones were transformed into DH10 Bac E. coli competent cells (Invitrogen) containing bacmid and helper. E. coli colonies with recombinant bacmid were screened by streaking on agar plates containing Blue-gal and relevant antibiotics. Positive colonies are white in color. The white colonies were restreaked to confirm the presence of recombinant bacmid. Recombinant bacmid DNA was isolated and transfected into Sf9 insect cells (Invitrogen) using Cellfectin Reagent (Invitrogen) (a detailed protocol is available in the Bac-to-Bac Baculovirus Expression System Manual from Invitrogen). Recombinant baculoviral stocks were isolated (after centrifugation and filtration through 0.45 μm filters) and the titer was calculated. The virus was then used to transduce human proximal tubule cells (HPTCs) using various multiplicities of infection (MOIs).

### Example 4

This example demonstrates lentiviral cloning of BMP-7.

Lentiviral Vector expressing BMP-7 under CMV promoter was purchased from GeneCopoeia, Inc. (Rockville, Md., USA) (Catalogue # EX-A0309-Lv105). SEQ ID NO. 2 is the nucleic acid sequence of BMP-7 in this vector.

The clones are available in the form of filter paper discs, which were incubated in 50 μl of water for one hour and transformed into One Shot® Stbl3™ Chemically Competent E. coli (Invitrogen, CA, USA) as described in the GeneCopoeia Transformation Protocol for cDNA clones. Colonies were screened using PCR and DNA sequencing. A positive clone was transfected into human embryonic kidney (HEK) 293T cells (packaging cell line, ATCC, VA, USA) using EndoFectin Lenti transfection reagent from GeneCopoeia Inc. (Rockville, Md., USA). The transfection was carried out according to manufacturer's instructions as described in the Lenti-Pac™ HIV Expression Packaging Kit user manual (GeneCopoeia Inc. (Rockville, Md., USA). Briefly, the packaging cells were incubated with DNA EndoFectin lenti complex and HIV packaging mix at 37° C. in a CO₂ incubator. Following overnight incubation, the culture media was replaced with fresh media supplemented with 5% fetal bovine serum and 1/500 volume TiterBoost (included in the kit). Incubation was continued for another 48 hours and pseudovirus-containing culture medium was collected and centrifuged to remove cell debris. Finally, the supernatant was filtered through 0.45 μm polyethersulphone low-protein binding filters. Aliquots of the virus were stored at −80° C.

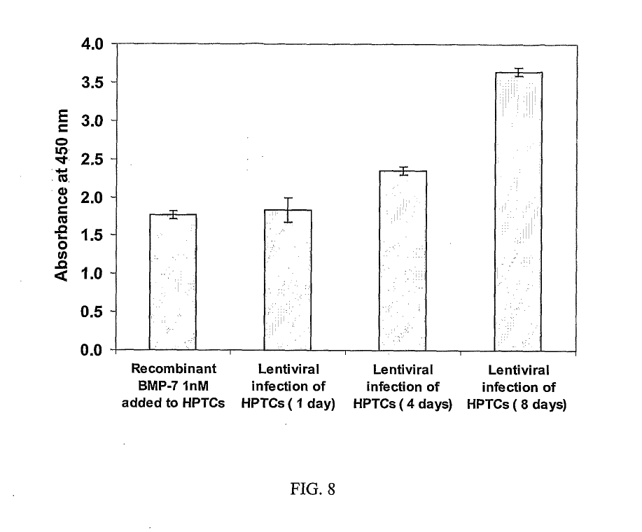

Human primary proximal renal tubular cells (HPTCs) were transduced with the virus. The dilution of the amount of virus (in the media) was optimized such that the BMP-7 produced was at the same level as cells treated with 25 ng/ml (1 nM) human recombinant BMP-7. The BMP-7 levels were measured by enzyme-linked immunosorbent assay (ELISA) (FIG. 8). FIG. 8 shows that at a dilution of 1:10 (virus:media), the BMP-7 produced by the virus in 1 day is similar to the level of BMP-7 level when Recombinant BMP-7 is added to the HPTCs. There is an increase in BMP-7 levels if the transduced cells are allowed to grow for 4 days and 8 days respectively.

### Example 5

This example demonstrates gamma-glutaryltransferase and hormone response assays.

Both gamma-glutaryltransferase (GGT) and hormone response assays were carried out in the mini-bioreactor. The mini-bioreactor is essentially a small bioreactor with two chambers (upper chamber and lower chamber) separated by a polysulfone-fullcure (PSFC) membrane. Cell culture media is perfused from a reservoir connected to the mini-bioreactor with the aid of a pump and tubings. HPTCs are seeded into the upper chamber through three-way-taps connected to the tubings. The cells are then allowed to attach to the membrane surface overnight before perfusion is started.

HPTCs were obtained from American Type Culture Collection (ATCC, Manassas, Va. USA) and cultivated in renal epithelial cell basal media supplemented with 0.5% fetal bovine serum (FBS), 1% penicllin/streptomycin and the renal cell growth kit (all components from ATCC).

Control HPTCs in the bioreactor were cultured in the media mentioned above. For BMP-7 treated cells, human recombinant BMP-7 (Miltenyi) was added at a concentration of 25 ng/ml (1 nM) to the media in the reservoir (inlet). The HPTCs in the bioreactor were perfused for four days in all cases.

Glutamyl transferase (GGT) activity was determined as described (Meister, A., S. S. Tate, and O. W. Griffith. 1981. Gamma-glutamyl transpeptidase. Methods Enzymol. 77:237-53), and the results are shown in FIG. 2. HPTCs in the mini-bioreactor were perfused with media (at the inlet) containing substrates for the reaction—1 mM γ-glutamyl-p-nitroanilide (Sigma) and 20 mM Glycyl-glycine (Sigma) for four hours (conditioning period). The flow-through coming out of the bioreactor was collected at a separate reservoir (outlet). Following the conditioning period, the reservoir at the outlet was discarded and replaced with a fresh empty reservoir. HPTCs were then incubated with media containing the substrates for one hour (assay period). The media from the inlet and the outlet was collected and the absorbance was measured at 405 nm using a microplate reader. GGT activity in cells was calculated from the standard curve (plotted using known concentrations (μmol/ml) of 4-nitroanaline (Merck)). Since the HPTCs were incubated for one hour, the GGT activity is presented as production of 4-nitroanaline μmol/ml/hr.

Hormone response in HPTCs was determined by overnight incubation of cells with medium containing 0.1 mM 3-isobutyl-1-methylxanthine (IBMX) (Wieser, M., G. Stadler, P. Jennings, B. Streubel, W. Pfaller, P. Ambros, C. Riedl, H. Katinger, J. Grillari, and R. Grillari-Voglauer. 2008. hTERT alone immortalizes epithelial cells of renal proximal tubules without changing their functional characteristics. Am J Physiol Renal Physiol. 295:F1365-75) and exposure of cells to 100 nmol/l of parathyroid hormone (PTH) for 3 hours at 37° C. (Control cells in the first bar in FIG. 1 were not exposed to PTH whereas control cells in the second bar in FIG. 1 were exposed to PTH). The cells were lysed and the intracellular concentration of cyclic adenosine monophosphate (cAMP) was determined using cAMP direct immunoassay kit (Calbiochem, affiliate of Merck). The Bradford method was used to quantify the amounts of proteins in cell extracts.

For both the assays, Excel 2003 was used for all calculations and statistics (unpaired t-test).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
contacting a plurality of renal proximal tubule cells in a fluidic device with sufficient BMP-7 or functional variants or functional fragments thereof and/or a BMP-7 agonist to inhibit tubule formation and/or improve cell performance by the plurality of renal proximal tubule cells.

2. The method of claim 1, comprising contacting a plurality of renal proximal tubule cells in a fluidic device with sufficient BMP-7 or functional variants or functional fragments thereof to inhibit tubule formation.

3. The method of claim 1, wherein the renal proximal tubule cells are genetically modified to overexpress the BMP-7 or functional variants or functional fragments thereof and/or the BMP-7 agonist.

4. The method of claim 1, wherein the renal proximal tubule cells are genetically modified to overexpress the BMP-7 or functional variants or functional fragments thereof.

5. The method of claim 2, wherein the plurality of renal proximal tubule cells are contacted with BMP-7 or functional variants or functional fragments thereof.

6. The method of claim 5, wherein the plurality of renal proximal tubule cells are contacted with BMP-7.

7. The method of claim 5, wherein the BMP-7 or functional variants or functional fragments thereof is present in a concentration of at least 0.5 nM.

8. The method of claim 1, wherein the plurality of renal proximal tubule cells are contacted with a BMP-7 agonist.

9. The method of claim 8, wherein the BMP-7 agonist is an isoform of KCP or functional variants or functional fragments thereof.

10. The method of claim 1, wherein the plurality of renal proximal tubule cells are residing on a semi-permeable membrane.

11. The method of claim 10, wherein the plurality of renal proximal tubule cells form a monolayer on the semi-permeable membrane.

12. The method of claim 1, wherein the fluidic device is an extracorporeal device for treating blood from a patient.

13. The method of claim 10, wherein the fluidic device is a bioartificial kidney comprising an ultrafiltration unit and a reabsorption unit, the reabsorption unit comprising the semi-permeable membrane.

14. The method of claim 13, wherein the plurality of renal proximal tubule cells are residing on a surface of a hollow fiber membrane.

15. The method of claim 14, wherein the surface is an inner surface of the hollow fiber membrane.

16. The method of claim 1, wherein the fluidic device comprises least one renal cell type selected from the group consisting of distal tubule cells, collecting duct cells, podocytes, cells of the thick ascending limb, and fibroblasts.

17. The method of claim 16, wherein the at least one renal cell expresses BMP-7 or functional variants or functional fragments thereof and/or a BMP-7 agonist.

18. The method of claim 1, wherein the fluidic device comprises renal fibroblasts.

19. The method of claim 18, wherein the renal fibroblasts express erythropoietin.

20. A method, comprising:
contacting a plurality of renal proximal tubule cells in a fluidic device with sufficient BMP-7 or functional variants or functional fragments thereof and/or a sufficient amount of a BMP-7 agonist to inhibit de-differentiation of the renal proximal tubule cells.

21. The method of claim 20, wherein the renal proximal tubule cells are genetically modified to overexpress the BMP-7 or functional variants or functional fragments thereof and/or the BMP-7 agonist.

22. The method of claim 20, wherein the renal proximal tubule cells are genetically modified to overexpress the BMP-7 or functional variants or functional fragments thereof.

23. The method of claim 20, wherein the renal proximal tubule cells are contacted with BMP-7 or functional variants or functional fragments thereof.

24. The method of claim 23, wherein the renal proximal tubule cells are contacted with BMP-7.

25. The method of claim 20, wherein the BMP-7 or functional variants or functional fragments thereof is present in a concentration of at least 0.5 nM.

26. The method of claim 20, wherein the plurality of renal proximal tubule cells are contacted with a BMP-7 agonist.

27. The method of claim 26, wherein the BMP-7 agonist is an isoform of KCP or functional variants or functional fragments thereof.

28. The method of claim 20, wherein the renal proximal tubule cells reside on a semi-permeable membrane.

29. The method of claim 20, wherein the fluidic device is an extracorporeal device for treating blood from a patient.

30. The method of claim 28, wherein the fluidic device is a bioartificial kidney comprising an ultrafiltration unit and a reabsorption unit, the reabsorption unit comprising the semi-permeable membrane.

31. The method of claim 30, wherein the renal proximal tubule cells reside on a surface of a hollow fiber membrane.

32. The method of claim 31, wherein the surface is an inner surface of the hollow fiber membrane.

33. The method of claim 20, wherein the fluidic device comprises least one renal cell type selected from the group consisting of distal tubule cells, collecting duct cells, podocytes, cells of the thick ascending limb, and fibroblasts.

34. The method of claim 33, wherein the at least one renal cell expresses BMP-7 or functional variants or functional fragments thereof and/or a BMP-7 agonist.

35. The method of claim 20, wherein the fluidic device comprises renal fibroblasts.

36. The method of claim 35, wherein the renal fibroblasts express erythropoietin.

37. A method, comprising:
administering a therapeutic amount of BMP-7 or functional variants or functional fragments thereof and/or a BMP agonist systemically to a patient, wherein the BMP-7 or functional variants or functional fragments thereof and/or a BMP-7 agonist is generated essentially continuously from cells within a fluidic device comprising said cells in fluid communication with the patient.

38. The method of claim 37, wherein the fluidic device comprises a plurality of renal proximal tubule cells.

39. The method of claim COO, wherein the plurality of renal proximal tubule cells generate the BMP-7 or functional variants or functional fragments thereof and/or a BMP-7 agonist.

40. The method of claim 39, wherein the plurality of renal proximal tubule cells generate BMP-7 or functional variants or functional fragments thereof.

41. The method of claim 40, wherein the plurality of renal proximal tubule cells generate BMP-7.

42. The method of claim 37, wherein at least some of the cells are genetically modified in order to overexpress BMP-7 or functional variants or functional fragments thereof and/or a BMP-7 agonist.

43. The method of claim 42, wherein at least some of the cells are genetically modified with an expression vector comprising a nucleic acid sequence coding for BMP-7 or a functional variant or functional fragment thereof.

44. The method of claim 43, wherein at least some of the cells are genetically modified with an expression vector comprising a nucleic acid sequence coding for BMP-7.

45. The method of claim 44, wherein the expression vector comprises a nucleic acid molecule that hybridizes to the nucleic acid sequence set forth in SEQ ID NO: 2 under high stringency conditions, and degenerates, complements, and unique fragments thereof.

46. The method of claim 37, wherein the cells reside on a semi-permeable membrane.

47. The method of claim COO, wherein the plurality of renal proximal tubule cells are contacted with BMP-7.

48. The method of claim 37, wherein the BMP-7 or functional variants or functional fragments thereof has a concentration of at least 0.5 nM.

49. The method of claim 37, wherein the plurality of renal proximal tubule cells are contacted with a BMP-7 agonist.

50. The method of claim 49, wherein the BMP-7 agonist is an isoform of KCP or functional variants or functional fragments thereof.

51. The method of claim 46, wherein the cells form a monolayer on the semi-permeable membrane.

52. The method of claim 37, wherein the fluidic device is an extracorporeal device for treating blood from a patient.

53. The method of claim 46, wherein the fluidic device is a bioartificial kidney comprising an ultrafiltration unit and a reabsorption unit, the reabsorption unit comprising the semi-permeable membrane.

54. The method of claim 38, wherein the plurality of renal proximal tubule cells reside on a surface of a hollow fiber membrane.

55. The method of claim 43, wherein the expression vector comprising the nucleic acid sequence is operably linked to a promoter.

56. The method of claim 45, wherein the expression vector comprising the nucleic acid molecule or degenerate or complement thereof is operably linked to a promoter.

57. The method of claim 37, wherein the cells comprise at least one renal cell type selected from the group consisting of distal tubule cells, collecting duct cells, podocytes, cells of the thick ascending limb, and fibroblasts.

58. The method of claim 57, wherein the cells further comprise renal proximal tubule cells.

59. The method of claim 37, wherein the cells comprise renal fibroblasts.

60. The method of claim 59, wherein the renal fibroblasts express erythropoietin.

61. The method of claim 42, wherein at least some of the cells are genetically modified with an expression vector comprising a nucleic acid sequence coding for an isoform of KCP or a functional variant or functional fragment thereof.

62. The method of claim 61, wherein at least some of the cells are genetically modified with an expression vector comprising a nucleic acid sequence coding for an isoform of KCP.

63. The method of claim 62, wherein the expression vector comprises a nucleic acid molecule that hybridizes to the nucleic acid sequence set forth in SEQ ID NO: 4 or SEQ ID NO: 6 under high stringency conditions, and degenerates, complements, and unique fragments thereof.

64. An apparatus, comprising:
a fluidic device comprising a plurality of host cells genetically modified for overexpression of BMP-7 or functional variants or functional fragments thereof and/or a BMP-7 agonist.

65. The apparatus of claim 64, wherein the host cells are renal proximal tubule cells.

66. The apparatus of claim 64, wherein the fluidic device is a bioartificial kidney.

67. The apparatus of claim 64, wherein at least some of the cells are genetically modified with an expression vector comprising a nucleic acid sequence coding for BMP-7 or functional variants or functional fragments thereof.

68. The apparatus of claim 67, wherein at least some of the cells are genetically modified with an expression vector comprising a nucleic acid sequence coding for BMP-7.

69. The apparatus of claim 68, wherein the expression vector comprises a nucleic acid molecule that hybridizes to the nucleic acid sequence set forth in SEQ ID NO: 2 under high stringency conditions, and degenerates, complements, and unique fragments thereof.

70. The apparatus of claim 64, wherein the cells reside on a semi-permeable membrane.

71. The apparatus of claim 70, wherein the cells form a monolayer on the semi-permeable membrane.

72. The apparatus of claim 64, wherein the fluidic device is an extracorporeal device for treating blood from a patient.

73. The apparatus of claim 66, wherein the bioartificial kidney comprises an ultrafiltration unit and a reabsorption unit, the reabsorption unit comprising a semi-permeable membrane.

74. The apparatus of claim 73, wherein the cells reside on a surface of a hollow fiber membrane.

75. The apparatus of claim 67, wherein the expression vector comprising the nucleic acid sequence is operably linked to a promoter.

76. The apparatus of claim 69, wherein the expression vector comprising the nucleic acid sequence or degenerate or complement thereof is operably linked to a promoter.

77. The apparatus of claim 64, wherein at least some of the cells are genetically modified with an expression vector comprising a nucleic acid sequence coding for an isoform of KCP or a functional variant or functional fragment thereof.

78. The apparatus of claim 77, wherein at least some of the cells are genetically modified with an expression vector comprising a nucleic acid sequence coding for an isoform of KCP.

79. The apparatus of claim 78, wherein the expression vector comprises a nucleic acid molecule that hybridizes to the nucleic acid sequence set forth in SEQ ID NO: 4 or SEQ ID NO: 6 under high stringency conditions, and degenerates, complements, and unique fragments thereof.

80. An apparatus, comprising:
a fluidic device comprising a semi-permeable membrane, wherein a non-cellular component of the apparatus is configured for controlled release of BMP-7 or functional variants or functional fragments thereof and/or a BMP-7 agonist.

81. The apparatus of claim 80, further comprising renal proximal tubule cells seeded on the semi-permeable membrane.

82. The apparatus of claim 80, wherein the fluidic device is a hemodialysis device.

83. The apparatus of claim 80, wherein the semi-permeable membrane is configured for controlled release of BMP-7 or functional variants or functional fragments thereof and/or a BMP-7 agonist.

84. The apparatus of claim 80, wherein the semi-permeable membrane comprises a plurality of particles configured for controlled release of BMP-7 or functional variants or functional fragments thereof and/or a BMP-7 agonist.

85. The apparatus of claim 80, wherein the non-cellular component of the apparatus is configured for controlled release of BMP-7 or functional variants or functional fragments thereof.

86. The apparatus of claim 85, wherein the non-cellular component of the apparatus is configured for controlled release of BMP-7.

87. The apparatus of claim 80, wherein the non-cellular component of the apparatus is configured for controlled release of a BMP-7 agonist.

88. The apparatus of claim 87, wherein the BMP-7 agonist is an isoform of ICP or functional variants or functional fragments thereof.

89. The apparatus of claim 80, wherein the fluidic device is an extracorporeal device for treating blood from a patient.

90. The apparatus of claim 80, wherein the fluidic device is a bioartificial kidney comprising an ultrafiltration unit and a reabsorption unit, the reabsorption unit comprising the semi-permeable membrane.

91. The apparatus of claim 80, further comprising at least one renal cell type selected from the group consisting of distal tubule cells, collecting duct cells, podocytes, cells of the thick ascending limb, and fibroblasts.

92. The apparatus of claim 80, further comprising renal fibroblasts.

93. A method, comprising:
administering BMP-7 or functional variants or functional fragments thereof and/or a BMP-7 agonist systemically to a patient, wherein the BMP-7 is released from in controlled fashion from a non-cellular component within a fluidic device.

94. The method of claim 93, further comprising a plurality renal proximal tubule cells in fluid communication with the patient.

95. The method of claim 93, wherein the fluidic device is a hemodialysis device.

96. The method of claim 93, wherein the non-cellular component within the fluidic device comprises a semi-permeable membrane.

97. The method of claim 93, wherein the semi-permeable membrane comprises a plurality of particles configured for controlled release of BMP-7 or functional variants or functional fragments thereof and/or a BMP-7 agonist.

98. The method of claim 93, wherein the non-cellular component of the apparatus is configured for controlled release of BMP-7 or functional variants or functional fragments thereof.

99. The method of claim 98, wherein the non-cellular component of the apparatus is configured for controlled release of BMP-7.

100. The method of claim 93, wherein the non-cellular component of the apparatus is configured for controlled release of a BMP-7 agonist.

101. The method of claim 100, wherein the BMP-7 agonist is an isoform of ICP or functional variants or functional fragments thereof.

102. The method of claim 93, wherein the fluidic device is an extracorporeal device for treating blood from a patient.

103. The method of claim 96, wherein a plurality renal proximal tubule cells reside on the semi-permeable membrane.

104. The method of claim 103, wherein the fluidic device is a bioartificial kidney comprising an ultrafiltration unit and a reabsorption unit, the reabsorption unit comprising the semi-permeable membrane.

105. The method of claim 93, further comprising at least one renal cell type selected from the group consisting of distal tubule cells, collecting duct cells, podocytes, cells of the thick ascending limb, and fibroblasts.

106. The method of claim 93, further comprising renal fibroblasts.

107. A semi-permeable membrane comprising:
at least one material configured for controlled release of BMP-7 or functional variants or functional fragments thereof and/or a BMP-7 agonist.

108. The semi-permeable membrane of claim 107, wherein the at least one material comprises particles configured for controlled release of BMP-7 or functional fragments thereof and/or a BMP-7 agonist.

109. The semi-permeable membrane of claim 108, wherein the at least one material comprises particles configured for controlled release of BMP-7.

110. The semi-permeable membrane of claim 108, wherein the particles are encapsulated in the membrane.

111. The semi-permeable membrane of claim 107, wherein the at least one material configured is configured for controlled release of a BMP-7 agonist.

112. The semi-permeable membrane of claim 111, wherein the BMP-7 agonist is an isoform of KCP or functional variants or functional fragments thereof.

113. The method or apparatus of any one of claim 1-7, 10-25, 28-48, 51-60, 64-76, 80-86, 89-99, or 102-109, wherein the BMP-7 or functional variants or functional fragments thereof has the amino acid sequence set forth in SEQ ID NO. 1.

114. The method or apparatus of any one of claim 1-7, 10-25, 28-48, 51-60, 64-76, 80-86, 89-99, or 102-109, wherein the BMP-7 or functional variants or functional fragments thereof is coded for by a nucleic acid having the nucleic acid sequence set forth in SEQ ID NO. 2 and degenerates, complements, and unique fragments thereof.

115. The method or apparatus of any one of claim 1-7, 10-25, 28-48, 51-60, 64-76, 80-86, 89-99, or 102-109, wherein the BMP-7 or functional variants or functional fragments thereof is coded for by the complement of a nucleic acid that hybridizes to the nucleic acid sequence set forth in SEQ ID NO: 2 under high stringency conditions, and degenerates thereof, complements, and unique fragments.

116. The method or apparatus of any one of claim 1-7, 10-25, 28-48, 51-60, 64-76, 80-86, 89-99, or 102-109, wherein the BMP-7 or functional variants or functional fragments thereof has an amino acid sequence with at least 80% homology to the amino acid sequence set forth in SEQ ID NO. 1.

117. The method or apparatus of any one of claim 1-7, 10-25, 28-48, 51-60, 64-76, 80-86, 89-99, or 102-109 wherein the BMP-7 or functional variants or functional fragments thereof has an amino acid sequence with at least 90% homology to the amino acid sequence set forth in SEQ ID NO. 1.

118. The method or apparatus of any one of claim 1-7, 10-25, 28-48, 51-60, 64-76, 80-86, 89-99, or 102-109, wherein the BMP-7 or functional variants or functional fragments thereof has an amino acid sequence with at least 95% homology to the amino acid sequence set forth in SEQ ID NO. 1.

119. The method or apparatus of any one of claim 1-7, 10-25, 28-48, 51-60, 64-76, 80-86, 89-99, or 102-109, wherein the BMP-7 or functional variants or functional fragments thereof has an amino acid sequence with at least 99% homology to the amino acid sequence set forth in SEQ ID NO. 1.

120. The method or apparatus of any one of claim 1-7, 10-25, 28-48, 51-60, 64-76, 80-86, 89-99, or 102-109, wherein a nucleic acid molecule has been introduced into the cells that encodes the amino acid sequence set forth in SEQ ID NO: 1.
